(19) United States
(12) Patent Application Publication
(10) Pub. No.: US 2008/0293782 A1
(43) Pub. Date: Nov. 27, 2008

(54) 1,1,3-Trioxo-1,2,5-Thiadiazolidines and Their Use as Ptp-Ases Inhibitors

(76) Inventors: David Barnes, Waban, MA (US); Gary Mark Coppola, Budd Lake, NJ (US); Robert Edson Damon, Hopkinton, MA (US); Katsumasa Nakajima, Winchester, MA (US); Brian Christopher Raudenbush, Beacon Falls, CT (US); Travis Stams, Stow, MA (US); Sidney Wolf Topiol, Fair Lawn, NJ (US); Thalaththani Ralalage Vedananda, Shrewsbury, MA (US)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/096,421

(22) PCT Filed: Dec. 6, 2006
(86) PCT No.: PCT/US2006/046544

§ 371 Date: Jun. 6, 2008

Related U.S. Application Data
(60) Provisional application No. 60/748,493, filed on Dec. 8, 2005.

(51) Int. Cl. A61K 31/433; C07D 285/10; A61P 3/10; A61P 3/04; A61P 9/10; A61P 25/28; A61P 35/00

(52) U.S. Cl. 514/362; 548/135

(57) ABSTRACT

Compounds of the formula

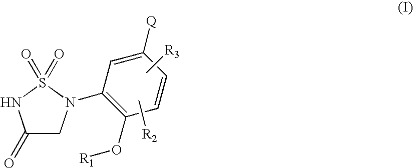

are inhibitors of protein tyrosine phosphatases (PTPases) and, thus, may be employed for the treatment of conditions mediated by PTPase activity. The compounds of the present invention may also be employed as inhibitors of other enzymes characterized with a phosphotyrosine binding region such as the SH2 domain. Accordingly, the compounds of formula (I) may be employed for prevention and/or treatment of insulin resistance associated with obesity, glucose intolerance, diabetes mellitus, hypertension and ischemic diseases of the large and small blood vessels, conditions that accompany type-2 diabetes, including hyperlipidemia, hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat and/or prevent cancer, osteoporosis, neurodegenerative and infectious diseases, and diseases involving inflammation and the immune system.

The present invention relates to thiadiazolidinone derivatives, pharmaceutical compositions containing such compounds, methods of making such and methods of treating conditions mediated by protein tyrosine phosphatases by employing such compounds.

Accordingly, the present invention provides compounds of the formula

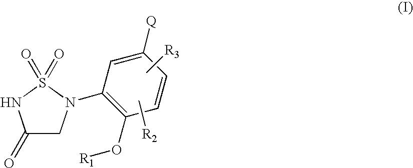

wherein
Q is alkoxy, alkylthio, alkylthiono, sulfonyl, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
   or a pharmaceutically acceptable salt thereof.

The compounds of the present invention are inhibitors of protein tyrosine phosphatases (PTPases), in particular, the compounds of formula (I) inhibit PTPase-1B (PTP-1B) and T-cell PTPase (TC PTP) and, thus, may be employed for the treatment of conditions mediated by PTPase activity. Accordingly, the compounds of formula (I) may be employed for treatment of insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer (such as prostate or breast cancer), osteoporosis, neurodegenerative and infectious diseases, and diseases involving inflammation and the immune system.

Listed below are definitions of various terms used to describe the compounds of the instant invention. These definitions apply to the terms as they are used throughout the specification unless they are otherwise limited in specific instances either individually or as part of a larger group. In general, whenever an alkyl group is referred to as a part of the structure, an optionally substituted alkyl is also intended.

Accordingly, the term “optionally substituted alkyl” refers to unsubstituted or substituted straight or branched chain hydrocarbon groups having 1 to 20 carbon atoms, preferably 1 to 8 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halogen, hydroxy, cycloalkyl, cycloalkoxy, acyl, acyloxy, alkoxy, alkyloxyalkoxy, alkanoyloxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaraloxy, heterocyclyl and heterocyclyloxy including indolyl, imidazolyl, furyl, thienyl, thiazolyl, pyrrolidyl, pyridyl, pyrimidyl, piperidyl, morpholinyl and the like.

The term “lower alkyl” refers to any of the above alkyl groups as described above having 1 to 7, preferably 1 to 4 carbon atoms.

The term “halogen” or “halo” refers to fluorine, chlorine, bromine and iodine.

The term “alkenyl” refers to any of the above alkyl groups having at least 2 carbon atoms and containing a carbon to carbon double bond at the point of attachment. Groups having 2 to 8 carbon atoms are preferred.

The term “alkynyl” refers to any of the above alkyl groups having at least two carbon atoms and containing a carbon to carbon triple bond at the point of attachment. Groups having 2 to 8 carbon atoms are preferred.

The term “alkylene” refers to a straight-chain bridge of 2-6, carbon atoms connected by single bonds, e.g., —(CH₂)x, wherein x is 2-6, which may be interrupted with one or more heteroatoms selected from O, S, S(O), S(O)₂ or NR″, wherein R″ may be hydrogen, alkyl, cycloalkyl, aryl, heterocyclyl, aralkyl, heteroaralkyl, acyl, carbamoyl, sulfonyl, alkoxycarbonyl, aryloxycarbonyl or aralkoxycarbonyl and the like; and the alkylene may further be substituted with one or more substituents selected from hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl, heterocyclyloxy and the like.

The term “cycloalkyl” refers to optionally substituted monocyclic, bicyclic or tricyclic hydrocarbon groups of 3 to 12 carbon atoms, each of which may be substituted by one or more substituents such as alkyl, halo, oxo, hydroxy, alkoxy, alkanoyl, acylamino, carbamoyl, alkylamino, dialkylamino, thiol, alkylthio, nitro, cyano, carboxy, carboxyalkyl, alkoxycarbonyl, sulfonyl, sulfonamido, sulfamoyl, heterocyclyl and the like.

Exemplary monocyclic hydrocarbon groups include but are not limited to cyclopropyl, cyclobutyl, cyclopentyl, cyclopentenyl, cyclohexyl and cyclohexenyl and the like.

Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like.

Exemplary tricyclic hydrocarbon groups include adamantyl and the like.

The term “alkoxy” refers to alkyl-O—.

The term “alkanoyl” refers to alkyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “alkylaminothiocarbonyl” refers to alkyl-NHC(S)—.

The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carboxycarbonyl” refers to HO—C(O)C(O)—.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “sulfamoyl” refers to H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

The term “sulfonamido” refers to alkyl-S(O)₂—NH—, aryl-S(O)₂—NH—, aralkyl-S(O)₂—NH—, heteroaryl-S(O)₂—NH—, heteroaralkyl-S(O)₂—NH—, alkyl-S(O)₂—N(alkyl)-, aryl-S(O)₂—N(alkyl)-, aralkyl-S(O)₂—N(alkyl)-, heteroaryl-S(O)₂—N(alkyl)-, heteroaralkyl-S(O)₂—N(alkyl)- and the like.

The term “sulfonyl” refers to alkylsulfonyl, arylsulfonyl, heteroarylsulfonyl, aralkylsulfonyl, heteroaralkylsulfonyl and the like.

The term “sulfonate” or “sulfonyloxy” refers to alkyl-S(O)₂—O—, aryl-S(O)₂—O—, aralkyl-S(O)₂—O—, heteroaryl-S(O)₂—O—, heteroaralkyl-S(O)₂—O— and the like.

The term “optionally substituted amino” refers to a primary or secondary amino group which may optionally be substituted by a substituent such as acyl, sulfonyl, alkoxycarbonyl, cycloalkoxycarbonyl, aryloxycarbonyl, heteroaryloxycarbonyl, aralkoxycarbonyl, heteroaralkoxycarbonyl, carboxycarbonyl, carbamoyl, alkylaminothiocarbonyl, arylaminothiocarbonyl and the like.

The term “aryl” refers to monocyclic or bicyclic aromatic hydrocarbon groups having 6 to 12 carbon atoms in the ring portion, such as phenyl, naphthyl, tetrahydronaphthyl, biphenyl and diphenyl groups, each of which may optionally be substituted by one to five substituents such as alkyl, trifluoromethyl, halo, hydroxy, alkoxy, acyl, alkanoyloxy, optionally substituted amino, thiol, alkylthio, nitro, cyano, carboxy, carboxyalkyl, alkoxycarbonyl, carbamoyl, alkylthiono, sulfonyl, sulfonamido, sulfonate, heterocyclyl and the like.

The term “monocyclic aryl” refers to optionally substituted phenyl as described under aryl.

The term “aralkyl” refers to an aryl group bonded directly through an alkyl group, such as benzyl.

The term “aralkanoyl” refers to aralkyl-C(O)—.

The term “aralkylthio” refers to aralkyl-S—.

The term “aralkoxy” refers to an aryl group bonded directly through an alkoxy group.

The term “arylsulfonyl” refers to aryl-S(O)₂—.

The term “arylthio” refers to aryl-S—.

The term “aroyl” refers to aryl-C(O)—.

The term “aroylamino” refers to aryl-C(O)—NH—.

The term “aryloxycarbonyl” refers to aryl-O—C(O)—.

The term “heterocyclyl” or “heterocyclo” refers to an optionally substituted, aromatic, or a partially or fully saturated nonaromatic cyclic group, for example, which is a 4- to 7-membered monocyclic, 7- to 12-membered bicyclic, or 10- to 15-membered tricyclic ring system, which has at least one heteroatom in at least one carbon atom-containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, benzodiazepinyl, cinnolinyl, quinoxalinyl, indazolyl, pyrrolopyridyl, furopyridinyl (such as furo[2,3-c]pyridinyl, furo[3,2-b]-pyridinyl] or furo[2,3-b]pyridinyl), dihydroisoindolyl, 1,3-dioxo-1,3-dihydroisoindol-2-yl, dihydroquinazolinyl (such as 3,4-dihydro-4-oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” includes substituted heterocyclic groups. Substituted heterocyclic groups refer to heterocyclic groups that are substituted with 1, 2 or 3 substituents selected from the group consisting of the following:
(a) optionally substituted alkyl;
(b) hydroxy (or protected hydroxy);
(c) halo;
(d) oxo (i.e. ═O);
(e) optionally substituted amino, alkylamino or dialkylamino;
(f) alkoxy;
(g) cycloalkyl;
(h) carboxy;
(i) heterocyclooxy;
(j) alkoxycarbonyl, such as unsubstituted lower alkoxycarbonyl;
(k) mercapto;
(l) nitro;
(m) cyano;
(n) sulfamoyl or sulfonamido;
(o) alkylcarbonyloxy;
(p) arylcarbonyloxy;
(q) arylthio;
(r) aryloxy;
(s) alkylthio;
(t) formyl;
(u) carbamoyl;
(v) aralkyl; and
(w) aryl substituted with alkyl, cycloalkyl, alkoxy, hydroxy, amino, acylamino, alkylamino, dialkylamino or halo.

The term “heterocyclooxy” denotes a heterocyclic group bonded through an oxygen bridge.

The term “heteroaryl” refers to an aromatic heterocycle, for example monocyclic or bicyclic aryl, such as pyrrolyl, pyrazolyl, imidazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by e.g. lower alkyl, lower alkoxy or halo.

The term “heteroarylsulfonyl” refers to heteroaryl-S(O)₂—.

The term “heteroaroyl” refers to heteroaryl-C(O)—.

The term “heteroaroylamino” refers to heteroaryl-C(O)NH—

The term “heteroaralkyl” refers to a heteroaryl group bonded through an alkyl group.

The term “heteroaralkanoyl” refers to heteroaralkyl-C(O)—.

The term “heteroaralkanoylamino” refers to heteroaralkyl-C(O)NH—.

The term “acyl” refers to alkanoyl, cycloalkanoyl, aroyl, heteroaroyl, aralkanoyl, heteroaralkanoyl and the like.

The term “acyloxy” refers to alkanoyloxy, cycloalkanoyloxy, aroyloxy, heteroaroyloxy, aralkanoyloxy, heteroaralkanoyloxy and the like.

The term “acylamino” refers to alkanoylamino, cycloalkanoylamino, aroylamino, heteroaroylamino, aralkanoylamino, heteroaralkanoylamino and the like.

The term “esterified carboxy” refers to optionally substituted alkoxycarbonyl, cycloalkoxycarbonyl, aryloxycarbonyl, aralkoxycarbonyl, heterocyclooxycarbonyl and the like.

Pharmaceutically acceptable salts of any compound of the present invention refer to salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris(hydroxymethyl)-methyl-ammonium salts, and salts with amino acids.

Similarly acid addition salts, such as those formed with mineral acids, organic carboxylic acids and organic sulfonic acids e.g. hydrochloric acid, maleic acid and methanesulfonic acid, are possible provided a basic group, such as pyridyl, constitutes part of the structure.

As described herein above, the present invention provides 1,1-dioxo-1,2,5-thiadiazolidin-3-one derivatives of formula (I), pharmaceutical compositions containing the same, methods for preparing such compounds and methods of treating and/or preventing conditions associated with PTPase activity, in particular, PTP-1B and TC PTP activity, by administration of a therapeutically effective amount of a compound of the present invention, or a pharmaceutical composition thereof.

Preferred are the compounds of formula (I), designated as the A group, wherein
Q is —Y—(CH₂)ₙ—CR₈R₉—(CH₂)ₘ—X in which
   Y is oxygen or S(O)_{q} in which q is zero or an integer of 1 or 2; or
   Y is C≡C; or
   Y is absent;
   n and m are, independently from each other, zero or an integer from 1 to 8;
   R₈ and R₉ are, independently from each other, hydrogen or lower alkyl; or
   R₈ and R₉ combined are alkylene which together with the carbon atom to which they are attached form a 3- to 7-membered ring;
   X is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
      or a pharmaceutically acceptable salt thereof.

Preferred are the compounds in the A group wherein
R₂ and R₃ are hydrogen;
   or a pharmaceutically acceptable salt thereof.

Further preferred are the compounds in the A group wherein
n is zero or an integer from 1 to 3;
m is zero or 1;
R₈ and R₉ are, independently from each other, hydrogen or lower alkyl;
X is hydroxy, carbamoyl, cyano, trifluoromethyl, free or esterified carboxy, heterocyclyl, monocyclic aryl or monocyclic aryloxy;
   or a pharmaceutically acceptable salt thereof.

Especially preferred are the compounds in the A group, designated as the B group, wherein
Y is C≡C; or
Y is absent;
   or a pharmaceutically acceptable salt thereof.

Preferred are the compounds in the B group wherein
Y is absent;
n is an integer of 5 or 6;
m is zero or 1;
R₈ and R₉ are lower alkyl;
X is hydroxy, cyano or free or esterified carboxy;
   or a pharmaceutically acceptable salt thereof.

Further preferred are the compounds in the B group wherein
R₈ and R₉ are methyl;
   or a pharmaceutically acceptable salt thereof.

Especially preferred are the compounds in the B group wherein
R₁ is hydrogen or —C(O)R₄ in which R₄ is monocyclic aryl;
   or a pharmaceutically acceptable salt thereof.

Preferred are also the compounds in the B group, designated as the C group, wherein
Y is absent;
n is an integer of 4 or 5;
m is zero;
R₈ and R₉ are hydrogen;
X is monocyclic aryloxy;
   or a pharmaceutically acceptable salt thereof.

Preferred are the compounds in the C group wherein
R₁ is hydrogen or —C(O)R₄ in which R₄ is monocyclic aryl;
   or a pharmaceutically acceptable salt thereof.

Preferred are also the compounds in the B group, designated as the D group, wherein
Y is C≡C;
n is an integer of 2 or 3;
m is zero;
R₈ and R₉ are hydrogen;
X is hydroxy, cyano or free or esterified carboxy;
   or a pharmaceutically acceptable salt thereof.

Preferred are the compounds in the D group wherein
R₁ is hydrogen or —C(O)R₄ in which R₄ is monocyclic aryl;
   or a pharmaceutically acceptable salt thereof.

Preferred are the compounds of formula (I), designated as the E group, wherein
Q is monocyclic aryl or 5- to 6-membered heterocyclic ring;
   or a pharmaceutically acceptable salt thereof.

Preferred are the compounds in the E group, designated as the G group, wherein
R₂ and R₃ are hydrogen;
   or a pharmaceutically acceptable salt thereof.

Preferred are the compounds in the G group having the formula

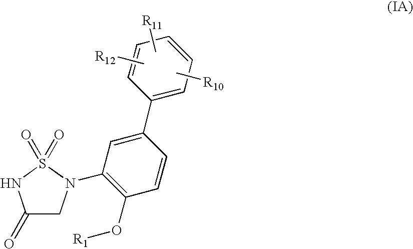

wherein
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₀, R₁₁ and R₁₂ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
C—R₁₀, C—R₁₁ and C—R₁₂ are, independently from each other, replaced by nitrogen;
   or a pharmaceutically acceptable salt thereof.

Preferred are the compounds of formula (IA) wherein
R₁₀ and R₁₁ are hydrogen;
   or a pharmaceutically acceptable salt thereof.

Preferred are also the compounds of formula (IA) wherein
R₁ is hydrogen or —C(O)R₄ in which R₄ is monocyclic aryl;
   or a pharmaceutically acceptable salt thereof.

Preferred are also the compounds in the G group having the formula

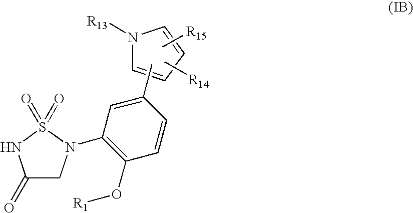

wherein
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁₄ and R₁₅ are, independently from each other, hydrogen or lower alkyl; or
C—R₁₄ and C—R₁₅ are, independently from each other, replaced by nitrogen;
   or a pharmaceutically acceptable salt thereof.

Preferred are the compounds of formula (IB) wherein
C—R₁₄ is replaced by nitrogen;
R₁₅ is hydrogen;
   or a pharmaceutically acceptable salt thereof.

Further preferred are the compounds of formula (IB) having the formula

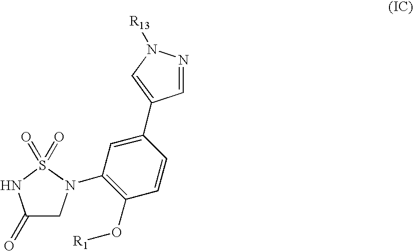

wherein
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Preferred are the compounds of formula (IC) wherein
R₁₃ is —(CH₂)ₙ—CR₁₆R₁₇—(CH₂)ₘ-Z in which
   n and m are, independently from each other, zero or an integer from 1 to 6;
   R₁₆ and R₁₇ are, independently from each other, hydrogen or lower alkyl; or
   R₁₆ and R₁₇ combined are alkylene which together with the carbon atom to which they are attached form a 3- to 7-membered ring;
   Z is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
      or a pharmaceutically acceptable salt thereof.

Further preferred are the compounds of formula (IC) wherein
n is an integer from 1 to 3;
m is zero or 1;
R₁₆ and R₁₇ are, independently from each other, hydrogen or lower alkyl;
Z is hydroxy, carbamoyl, cyano, trifluoromethyl, free or esterified carboxy, heterocyclyl, monocyclic aryl or monocyclic aryloxy;
   or a pharmaceutically acceptable salt thereof.

More preferred are the compounds of formula (IC) wherein
R₁₆ and R₁₇ are hydrogen;
Z is hydroxy, cyano or free or esterified carboxy;
   or a pharmaceutically acceptable salt thereof.

Most preferred are the compounds of formula (IC) wherein
R₁ is hydrogen or —C(O)R₄ in which R₄ is monocyclic aryl;
   or a pharmaceutically acceptable salt thereof.

Particular embodiments of the invention are:
5-[2-Hydroxy-5-(1H-pyrrol-2-yl)-phenyl]-1,1-dioxo-1,2,5-thiadiazolidin-3-one;
5-(4-Hydroxybiphenyl-3-yl)-1,1-dioxo-1,2,5-thiadiazolidin-3-one;
5-[2-Hydroxy-5-(2H-pyrazol-3-yl)-phenyl]-1,1-dioxo-1,2,5-thiadiazolidin-3-one;
5-[2-Hydroxy-5-(1-methyl-1H-pyrazol-4-yl)-phenyl]-1,1-dioxo-1,2,5-thiadiazolidin-3-one;
5-(5-Furan-3-yl-2-hydroxyphenyl)-1,1-dioxo-1,2,5-thiadiazolidin-3-one;
5-[2-Hydroxy-5-(1H-pyrazol-4-yl)-phenyl]-1,1-dioxo-1,2,5-thiadiazolidin-3-one;
5-(4′-Acetyl-4-hydroxybiphenyl-3-yl)-1,1-dioxo-1,2,5-thiadiazolidin-3-one;
5-(4′-Benzoyl-4-hydroxybiphenyl-3-yl)-1,1-dioxo-1,2,5-thiadiazolidin-3-one;
5-[2-Hydroxy-5-(1H-pyrrol-3-yl)-phenyl]-1,1-dioxo-1,2,5-thiadiazolidin-3-one;
Methanesulfonic acid 4′-hydroxy-3′-(1,1,4-trioxo-1,2,5-thiadiazolidin-2-yl)-biphenyl-3-yl ester;
5-(3′-Amino-4-hydroxybiphenyl-3-yl)-1,1-dioxo-1,2,5-thiadiazolidin-3-one;
5-(4-Hydroxy-2′-methylbiphenyl-3-yl)-1,1-dioxo-1,2,5-thiadiazolidin-3-one;
5-[2-Hydroxy-5-(1H-indol-2-yl)-phenyl]-1,1-dioxo-1,2,5-thiadiazolidin-3-one;
[4′-Hydroxy-3′-(1,1,4-trioxo-1,2,5-thiadiazolidin-2-yl)-biphenyl-3-yl]-acetonitrile;
4′-Hydroxy-3′-(1,1,4-trioxo-1,2,5-thiadiazolidin-2-yl)-biphenyl-3-carboxylic acid (2-cyanoethyl)-amide;
3-[4′-Hydroxy-3′-(1,1,4-trioxo-1,2,5-thiadiazolidin-2-yl)-biphenyl-3-yl]-propionic acid methyl ester;
4′-Hydroxy-3′-(1,1,4-trioxo-1,2,5-thiadiazolidin-2-yl)-biphenyl-3-carboxylic acid (2-carbamoylethyl)-amide;
5-[3′-(2-Aminoethyl)-4-hydroxybiphenyl-3-yl]-1,1-dioxo-1,2,5-thiadiazolidin-3-one;
5-(3′-Aminomethyl-4-hydroxybiphenyl-3-yl)-1,1-dioxo-1,2,5-thiadiazolidin-3-one;
5-(2-Hydroxy-5-pyridin-3-yl-phenyl)-1,1-dioxo-1,2,5-thiadiazolidin-3-one;
5-(4-Hydroxy-2′-methoxy-biphenyl-3-yl)-1,1-dioxo-1,2,5-thiadiazolidin-3-one;
5-(2-Hydroxy-5-pyridin-4-yl-phenyl)-1,1-dioxo-1,2,5-thiadiazolidin-3-one;
[4′-Hydroxy-3′-(1,1,4-trioxo-1,2,5-thiadiazolidin-2-yl)-biphenyl-4-yl]-acetic acid;
5-(4′-Chloro-4-hydroxybiphenyl-3-yl)-1,1-dioxo-1,2,5-thiadiazolidin-3-one;
5-(3′-Chloro-4-hydroxybiphenyl-3-yl)-1,1-dioxo-1,2,5-thiadiazolidin-3-one;
5-[2-Hydroxy-5-(6-methoxypyridin-3-yl)-phenyl]-1,1-dioxo-1,2,5-thiadiazolidin-3-one;
5-[5-(6-Fluoropyridin-3-yl)-2-hydroxyphenyl]-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionic acid ethyl ester;
5-(4-Hydroxy-3′-methylbiphenyl-3-yl)-1,1-dioxo-1,2,5-thiadiazolidin-3-one;
5-(3′-Fluoro-4-hydroxybiphenyl-3-yl)-1,1-dioxo-1,2,5-thiadiazolidin-3-one;
5-(4′-Fluoro-4-hydroxybiphenyl-3-yl-1,1-dioxo-1,2,5-thiadiazolidin-3-one;
5-(4-Hydroxy-4′-methylbiphenyl-3-yl)-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionitrile;
4′-Hydroxy-3′-(1,1,4-trioxo-1,2,5-thiadiazolidin-2-yl)-biphenyl-3-carbonitrile;
5-(4-Hydroxy-3′,5′-dimethylbiphenyl-3-yl)-1,1-dioxo-1,2,5-thiadiazolidin-3-one;
5-(4-Hydroxy-3′-methoxybiphenyl-3-yl)-1,1-dioxo-1,2,5-thiadiazolidin-3-one;
N-(2-Hydroxyethyl)-2-[4′-hydroxy-3′-(1,1,4-trioxo-1,2,5-thiadiazolidin-2-yl)-biphenyl-4-yl]-acetamide;
2,2,2-Trifluoro-N-[4′-hydroxy-3′-(1,1,4-trioxo-1,2,5-thiadiazolidin-2-yl)-biphenyl-3-yl]-acetamide;
1-Ethyl-3-[4′-hydroxy-3′-(1,1,4-trioxo-1,2,5-thiadiazolidin-2-yl)-biphenyl-3-yl]-urea;
1-Ethyl-3-[4′-hydroxy-3′-(1,1,4-trioxo-1,2,5-thiadiazolidin-2-yl)-biphenyl-3-ylmethyl]-urea;
[4′-Hydroxy-3′-(1,1,4-trioxo-1,2,5-thiadiazolidin-2-yl)-biphenyl-3-ylmethyl]-carbamic acid methyl ester;
N-[4′-Hydroxy-3′-(1,1,4-trioxo-1,2,5-thiadiazolidin-2-yl)-biphenyl-3-ylmethyl]-acetamide;
[4′-Hydroxy-3′-(1,1,4-trioxo-1,2,5-thiadiazolidin-2-yl)-biphenyl-3-ylmethyl]-carbamic acid benzyl ester;
1-Ethyl-3-[4′-hydroxy-3′-(1,1,4-trioxo-1,2,5-thiadiazolidin-2-yl)-biphenyl-4-yl]-urea;
3-[4′-Hydroxy-3′-(1,1,4-trioxo-1,2,5-thiadiazolidin-2-yl)-biphenyl-3-yl]-propionic acid;
5-{4-[4-Hydroxy-3-(1,1,4-trioxo-1,2,5-thiadiazolidin-2-yl)-phenyl]-pyrazol-1-yl}-pentanoic acid;
5-[2-Hydroxy-5-(1-propyl-1H-pyrazol-4-yl)-phenyl]-1,1-dioxo-1,2,5-thiadiazolidin-3-one;
5-[2-Hydroxy-5-(1-isobutyl-1H-pyrazol-4-yl)-phenyl]-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanoic acid ethyl ester;
5-{2-Hydroxy-5-[1-(4,4,4-trifluorobutyl)-1H-pyrazol-4-yl]-phenyl}-1,1-dioxo-1,2,5-thiadiazolidin-3-one;
5-{2-Hydroxy-5-[1-(3-methylbutyl)-1H-pyrazol-4-yl]-phenyl}-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanenitrile;
4-{4-[4-Hydroxy-3-(1,1,4-trioxo-1,2,5-thiadiazolidin-2-yl)-phenyl]-1H-pyrazol-1-yl}-butyronitrile;
5-(2-Hydroxy-5-phenoxyphenyl)-1,1-dioxo-1,2,5-thiadiazolidin-3-one;
5-(2-Hydroxy-5-methoxyphenyl)-1,1-dioxo-1,2,5-thiadiazolidin-3-one;
5-(5-Benzyl-2-hydroxyphenyl)-1,1-dioxo-1,2,5-thiadiazolidin-3-one;
5-(2-Hydroxy-5-methylphenyl)-1,1-dioxo-1,2,5-thiadiazolidin-3-one;
5-(5-Hexyl-2-hydroxyphenyl)-1,1-dioxo-1,2,5-thiadiazolidin-3-one;
5-(5-Butyl-2-hydroxyphenyl)-1,1-dioxo-1,2,5-thiadiazolidin-3-one;
5-[2-Hydroxy-5-(tetrahydrofuran-3-yl)-phenyl]-1,1-dioxo-1,2,5-thiadiazolidin-3-one;
5-[5-(4-Fluorophenylethynyl)-2-hydroxyphenyl]-1,1-dioxo-1,2,5-thiadiazolidin-3-one;
6-[4-Hydroxy-3-(1,1,4-trioxo-1,2,5-thiadiazolidin-2-yl)-phenyl]-hex-5-ynenitrile;
6-[4-Hydroxy-3-(1,1,4-trioxo-1,2,5-thiadiazolidin-2-yl)-phenyl]-hex-5-ynoic acid;
5-[5-(3,3-Dimethyl-but-1-ynyl)-2-hydroxyphenyl]-1,1-dioxo-1,2,5-thiadiazolidin-3-one;
5-[2-Hydroxy-5-(5-methylhexyl)-phenyl]-1,1-dioxo-1,2,5-thiadiazolidin-3-one;
6-[4-Hydroxy-3-(1,1,4-trioxo-1,2,5-thiadiazolidin-2-yl)-phenyl]-hexanoic acid;
5-[5-(Benzylaminomethyl)-2-hydroxyphenyl]-1,1-dioxo-1,2,5-thiadiazolidin-3-one;
5-(5-Butylaminomethyl-2-hydroxyphenyl)-1,1-dioxo-1,2,5-thiadiazolidin-3-one;
5-{2-Hydroxy-5-[(2-methoxybenzylamino)-methyl]-phenyl}-1,1-dioxo-1,2,5-thiadiazolidin-3-one;
5-{5-[(2-Ethoxybenzylamino)-methyl]-2-hydroxyphenyl}-1,1-dioxo-1,2,5-thiadiazolidin-3-one;
5-{2-Hydroxy-5-[(2-isopropoxybenzylamino)-methyl]-phenyl}-1,1-dioxo-1,2,5-thiadiazolidin-3-one;
5-(2-Hydroxy-5-{[2-(1-methyl-2-phenylethoxy)-benzylamino]-methyl}-phenyl)-1,1-dioxo-1,2,5-thiadiazolidin-3-one;
5-[2-Hydroxy-5-(3-methylbutoxy)-phenyl]-1,1-dioxo-1,2,5-thiadiazolidin-3-one;
5-[2-Hydroxy-5-(4-methylpentyloxy)-phenyl]-1,1-dioxo-1,2,5-thiadiazolidin-3-one;
5-(2-Hydroxy-5-propoxyphenyl)-1,1-dioxo-1,2,5-thiadiazolidin-3-one;
2-Hydroxy-6-{4-[4-hydroxy-3-(1,1,4-trioxo-1,2,5-thiadiazolidin-2-yl)-phenyl]-butoxy}-N,N-dimethylbenzamide;
2-Hydroxy-6-{5-[4-hydroxy-3-(1,1,4-trioxo-1,2,5-thiadiazolidin-2-yl)-phenyl]-pentyloxy}-N,N-dimethylbenzamide;
2-Hydroxy-6-{6-[4-hydroxy-3-(1,1,4-trioxo-1,2,5-thiadiazolidin-2-yl)-phenyl]-hexyloxy}-N,N-dimethylbenzamide;
2-Fluoro-6-{6-[4-hydroxy-3-(1,1,4-trioxo-1,2,5-thiadiazolidin-2-yl)-phenyl]-hexyloxy}-N,N-dimethylbenzamide;
2-Hydroxy-6-{7-[4-hydroxy-3-(1,1,4-trioxo-1,2,5-thiadiazolidin-2-yl)-phenyl]-heptyloxy}-N,N-dimethylbenzamide;
5-(4-Hydroxy-4′-hydroxymethylbiphenyl-3-yl)-1,1-dioxo-1,2,5-thiadiazolidin-3-one;
5-(2-Hydroxy-4,5-dimethylphenyl)-1,1-dioxo-1,2,5-thiadiazolidin-3-one;
5-[4-Hydroxy-3-(1,1,4-trioxo-1,2,5-thiadiazolidin-2-yl)-phenyl]-2,2-dimethylpentanoic acid;
8-[4-Hydroxy-3-(1,1,4-trioxo-1,2,5-thiadiazolidin-2-yl)-phenyl]-2,2-dimethyloctanoic acid ethyl ester;
8-[4-Hydroxy-3-(1,1,4-trioxo-1,2,5-thiadiazolidin-2-yl)-phenyl]-2,2-dimethyloctanoic acid;
7-[4-Hydroxy-3-(1,1,4-trioxo-1,2,5-thiadiazolidin-2-yl)-phenyl]-2,2-dimethylheptanoic acid;
6-[4-Hydroxy-3-(1,1,4-trioxo-1,2,5-thiadiazolidin-2-yl)-phenyl]-2,2-dimethylhexanoic acid;
7-[4-Hydroxy-3-(1,1,4-trioxo-1,2,5-thiadiazolidin-2-yl)-phenyl]-2,2-dimethylheptanoic acid ethyl ester;
8-[4-Hydroxy-3-(1,1,4-trioxo-1,2,5-thiadiazolidin-2-yl)-phenyl]-2,2-dimethyloctanenitrile;
5-[2-Hydroxy-5-(6-hydroxy-6-methylheptyl)-phenyl]-1,1-dioxo-1,2,5-thiadiazolidin-3-one;
5-[2-Hydroxy-5-(7-hydroxy-6,6-dimethylheptyl)-phenyl]-1,1-dioxo-1,2,5-thiadiazolidin-3-one;
5-[2-Hydroxy-5-(5-hydroxy-5-methylhexyl)-phenyl]-1,1-dioxo-1,2,5-thiadiazolidin-3-one;
5-[2-hydroxy-5-(8-hydroxy-7,7-dimethyloctyl)-phenyl]-1,1-dioxo-1,2,5-thiadiazolidin-3-one;
7-[4-Hydroxy-3-(1,1,4-trioxo-1,2,5-thiadiazolidin-2-yl)-phenyl]-2,2-dimethylheptanenitrile;
5-[2-Hydroxy-5-(5-hydroxy-5-methylhex-1-ynyl)-phenyl]-1,1-dioxo-1,2,5-thiadiazolidin-3-one;
5-[2-Hydroxy-5-(2-pyridin-3-yl-ethyl)-phenyl]-1,1-dioxo-1,2,5-thiadiazolidin-3-one;
5-(2-Hydroxy-4-methyl-5-pentylphenyl)-1,1-dioxo-1,2,5-thiadiazolidin-3-one;
5-(2-Hydroxy-4-methyl-5-propylphenyl)-1,1-dioxo-1,2,5-thiadiazolidin-3-one;
5-(5-Heptyl-2-hydroxy-4-methylphenyl)-1,1-dioxo-1,2,5-thiadiazolidin-3-one;
5-[5-(2-Cyclohexylethyl)-2-hydroxy-4-methylphenyl]-1,1-dioxo-1,2,5-thiadiazolidin-3-one;
Benzoic acid 4-(7-hydroxy-6,6-dimethylheptyl)-2-(1,1,4-trioxo-1,2,5-thiadiazolidin-2-yl)-phenyl ester; and
Benzoic acid 4-(6-cyano-6,6-dimethylhexyl)-2-(1,1,4-trioxo-1,2,5-thiadiazolidin-2-yl)-phenylester;
   or a pharmaceutically acceptable salt thereof.

The compounds of the invention depending on the nature of the substituents, may possess one or more asymmetric centers. The resulting diastereoisomers, enantiomers and geometric isomers are encompassed by the instant invention.

Compounds of formula (I) may be prepared starting, e.g., by cyclizing compounds of the formula

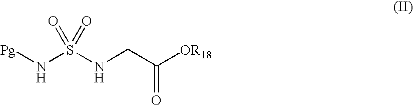

wherein Pg is an appropriate N-protecting group such as 4-methoxybenzyl, 2,4-dimethoxybenzyl or 2-trimethylsilylethyl, and R₁₈ is hydrogen to afford compounds of the formula

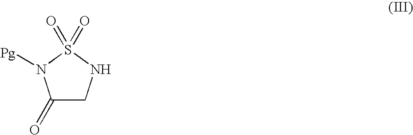

wherein Pg has a meaning as defined herein above, by treatment with a coupling agent such as diisopropyl carbodiimide (DIC) or 1-ethyl-3-(3-dimethylaminopropyl)carbodiimide hydrochloride (EDCI) in the presence a base such as triethylamine (TEA) or N-methyl-morpholine (NMM) in an organic solvent such as tetrahydrofuran (THF), N,N-dimethyl-formamide (DMF) or dichoromethane (DCM). The reaction may be carried out in the presence of an additive such as of hydroxybenzotriazole (HOBt).

Compounds of formula (II) wherein R₁₈ is hydrogen may be obtained from compounds of formula (II) wherein R₁₈ is an alkyl group according to methods well known in the art, e.g. compounds of formula (II) in which R₁₈ is methyl or ethyl can be treated with an aqueous base such as sodium or potassium hydroxide in an organic solvent such as THF, 1,4-dioxane, methanol (MeOH) or ethanol (EtOH) to afford compounds of formula (II) wherein R₁₈ is hydrogen, or compounds of formula (II) in which R₁₈ is t-butyl may be treated with an acid such as hydrochloric acid (HCl) or trifluoroacetic acid (TFA) in an organic solvent such as DCM or ethyl acetate (EtOAc) to afford compounds of formula (II) wherein R₁₈ is hydrogen.

Compounds of formula (II) wherein R₁₈ is an alkyl group such as methyl, ethyl or t-butyl, and the like, may be obtained analogously to a literature procedure described by Ducry et al. in Helvetica Chimica Acta, 1999, 82, 2432.

Resulting compounds of formula (III) wherein Pg has a meaning as defined herein can then be coupled with a variety of boronic acid derivatives of the formula

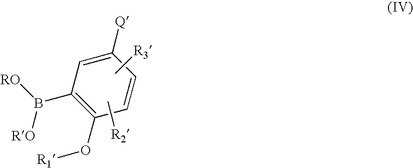

wherein R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, and R and R′ are hydrogen or lower alkyl, or R and R′ combined are alkylene which together with the boron and the oxygen atoms form a 5- or 6-membered ring, in the presence of a copper catalyst such as copper (II) acetate and a base such as cesium (II) carbonate (Cs₂CO₃) or TEA in an organic solvent such as THF, 1,4-dioxane or DCM to form compounds of the formula

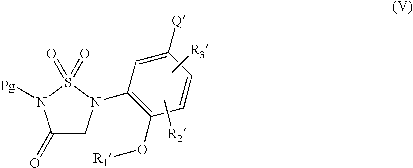

wherein Pg, R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively. Alternatively, compounds of formula (III) may be coupled with a boroxine derivative corresponding to a boronic acid derivative of formula (IV) as described, e.g., by Chan et al. in Tet. Lett. 2003, 44, 3863.

Compounds of formula (IV) are known, or if they are novel, they may be prepared using methods well known in the art, or as illustrated herein in the Examples, or modifications thereof.

Alternatively, compounds of formula (V) wherein R₁′, R₂′, R₃′, R₄′ and R₅′ have meanings as defined herein for R₁, R₂, R₃, R₄ and R₅, or R₁′, R₂′, R₃′, R₄′ and R₅′ are groups convertible to R₁, R₂, R₃, R₄ and R₅, respectively, may be obtained by reacting a compound of formula (III) wherein Pg has a meaning as defined herein with compounds of the formula

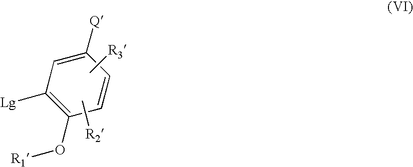

wherein Lg represents a leaving group such as halide or trifluoromethanesulfonate, preferably fluoride or chloride, and R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, using conditions well know in the art or using methods described herein or modifications thereof, e.g., a compound of formula (III) may be first treated with a base such as Cs₂CO₃, or sodium, lithium or potassium bis(trimethylsilyl) amide in an inert organic solvent such as THF or 1,4-dioxane followed by reaction with a compound of formula (VI) at a temperature ranging from room temperature (RT) to 110° C.

Compounds of formula (VI) are known, or if they are novel, they may be prepared using methods well known in the art, or as illustrated herein in the Examples, or modifications thereof.

Compounds of formula (V) wherein Pg, R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, can be converted to compounds of the formula

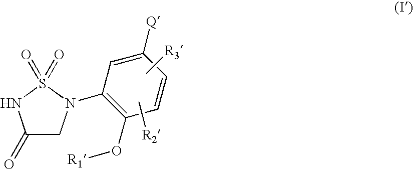

by removal of the N-protecting group according to methods well known in the art, e.g. in particular when Pg is 4-methoxybenzyl or 2,4-dimethoxybenzyl group using hydrogen in the presence of a catalyst such as palladium on carbon in a polar organic solvent such as MeOH or EtOAc, or by treatment with an acid such as TFA in an organic solvent such as DCM, preferably in the presence of an additive such as t-butyldimethylsilane or triethylsilane, or in particular when Pg is trimethylsilylethyl group using a fluoride reagent such as tetra-n-butylammoniumfluoride in an organic solvent such as THF or 1,4-dioxane.

In addition, compounds of formula (I′) wherein R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, may be prepared by condensing compounds of the formula

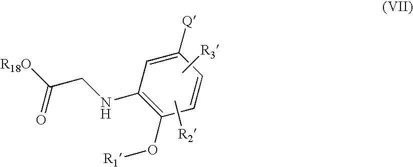

wherein R₁₈ has a meaning as defined herein above, with sulfamoyl chloride analogs of the formula

CIS(O)₂NHR₁₉ (VIII)

wherein R₁₉ is hydrogen or alkoxycarbonyl such as t-butoxycarbonyl or 2-trimethylsilyl-ethoxycarbonyl in the presence of a base such as TEA or NMM in an organic solvent such as acetonitrile (MeCN), DCM or THF to form compounds of the formula

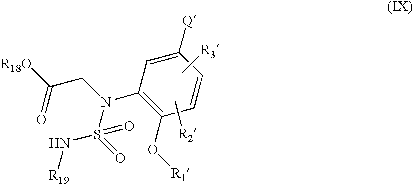

wherein R₁₈ and R₁₉ have meanings as defined herein, and R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively.

Compounds of formula (VIII) wherein R₁₉ is alkoxycarbonyl may be obtained by reacting chlorosulfonyl isocyanate with the appropriate alcohol in an organic solvent such as MeCN, DCM or THF.

Compounds of formula (VII) may be prepared using methods well known in the art or according to methods described herein or modifications thereof, e.g., under conditions of reductive amination, or according to the method described by Tohru Fukuyama et al. in Tet. Lett., 1997, 38 (33), 5831; or by reacting amines of the formula

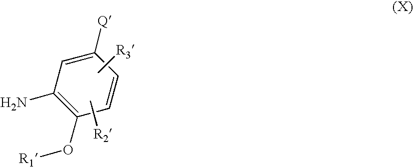

wherein R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, with an acetate of the formula

Lg′-CH₂—C(O)—O—R₁₈ (XI)

wherein Lg′ and R₁₈ have meanings as defined herein, in the presence of a base such as TEA or NMM in an inert solvent such as THF or 1,4-dioxane.

Amines of formula (X) are known, or if they are novel, they may be obtained according to methods well known in the art, or as described herein in the illustrative Examples, or using modifications thereof.

Compounds of formula (IX) wherein R₁₈ has a meaning as defined herein, and R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, and R₁₉ is alkoxycarbonyl may be converted to compounds of formula (IX) wherein R₁₉ is hydrogen according to methods known in the art or using methods described herein or modifications thereof, e.g., compounds of formula (IX) wherein R₁₉ is t-butoxycarbonyl may be treated with an acid such as TFA, neat or in an extrinsic organic solvent such as DCM, or compounds of formula (IX) wherein R₁₉ is 2-trimethylsilylethoxycarbonyl may be treated with a fluoride reagent such as tetra-n-butylammoniumfluoride in an organic solvent such as THF or 1,4-dioxane to afford compounds of formula (IX) wherein R₁₉ is hydrogen.

Compounds of formula (IX) wherein R₁₈ has a meaning as defined herein, and R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively, and R₁₉ is hydrogen can be cyclized to form compounds of formula (I′) using methods and conditions well known in the art or as illustrated with Examples herein or modifications thereof.

Alternatively, compounds of formula (IX) wherein R₁₈ has a meaning as defined herein; R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively; and R₁₉ is hydrogen, may be obtained by first condensing amines of formula (X) with sulfamide in an aqueous solution and in the presence of a base such as sodium bicarbonate (NaHCO₃) at an elevated temperature, preferably at the boiling point of the solution, to afford compounds of the formula

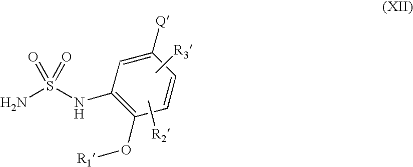

wherein R₁′, R₂′, R₃′ and Q′ have meanings as defined herein for R₁, R₂, R₃ and Q, or R₁′, R₂′, R₃′ and Q′ are groups convertible to R₁, R₂, R₃ and Q, respectively. Compound of formula (XII) may then be converted to compound of formula (IX) in which R₁₉ is hydrogen by the reaction with acetates of formula (XI) in the presence of a base such as sodium hydride in an inert solvent such as THF or DMF.

In starting compounds and intermediates which are converted to the compounds of the invention in a manner described herein, functional groups present, such as amino, thiol, carboxyl, and hydroxy groups, are optionally protected by conventional protecting groups that are common in preparative organic chemistry. Protected amino, thiol, carboxyl, and hydroxyl groups are those that can be converted under mild conditions into free amino thiol, carboxyl and hydroxyl groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxyl group, amino group, etc.), the structure and stability of the molecule of which the substituent is a part and the reaction conditions.

Well known protecting groups that meet these conditions and their introduction and removal are described, for example, in McOmie, “Protective Groups in Organic Chemistry”, Plenum Press, London, New York (1973); and Greene and Wuts, “Protective Groups in Organic Synthesis”, John Wiley and Sons, Inc, New York (1999).

The above mentioned reactions are carried out according to standard methods, in the presence or absence of diluent, preferably such as are inert to the reagents and are solvents thereof, of catalysts, condensing or said other agents respectively and/or inert atmospheres, at low temperatures, room temperature or elevated temperatures (preferably at or near the boiling point of the solvents used), and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known per se.

The invention also relates to any novel starting materials, intermediates and processes for their manufacture.

Depending on the choice of starting materials and methods, the new compounds may be in the form of one of the possible isomers or mixtures thereof, for example, as substantially pure geometric (cis or trans) isomers, optical isomers (enantiomers, antipodes), racemates, or mixtures thereof. The aforesaid possible isomers or mixtures thereof are within the purview of this invention.

Any resulting mixtures of isomers can be separated on the basis of the physico-chemical differences of the constituents, into the pure geometric or optical isomers, diastereoisomers, racemates, for example by chromatography and/or fractional crystallization.

Any resulting racemates of final products or intermediates can be resolved into the optical antipodes by known methods, e.g. by separation of the diastereoisomeric salts thereof, obtained with an optically active acid or base, and liberating the optically active acidic or basic compound. The carboxylic acid intermediates can thus be resolved into their optical antipodes e.g. by fractional crystallization of D- or L-(alpha-methylbenzylamine, cinchonidine, cinchonine, quinine, quinidine, ephedrine, dehydroabietylamine, brucine or strychnine)-salts. Racemic products can also be resolved by chiral chromatography, e.g. high pressure liquid chromatography using a chiral adsorbent.

Finally, compounds of the invention are either obtained in the free form, as a salt thereof if salt forming groups are present or as prodrug derivatives thereof.

In particular, the NH-group of the 1,1-dioxo-1,2,5-thiadiazolidin-3-one moiety, may be converted into salts with pharmaceutically acceptable bases. Salts may be formed using conventional methods, advantageously in the presence of an ethereal or alcoholic solvent, such as a lower alkanol. From the solutions of the latter, the salts may be precipitated with ethers, e.g. diethyl ether. Resulting salts may be converted into the free compounds by treatment with acids. These or other salts can also be used for purification of the compounds obtained.

Compounds of the invention having basic groups can be converted into acid addition salts, especially pharmaceutically acceptable salts. These are formed, for example, with inorganic acids, such as mineral acids, for example sulfuric acid, a phosphoric or hydrohalic acid, or with organic carboxylic acids, such as (C₁₋₄)alkanecarboxylic acids which, for example, are unsubstituted or substituted by halogen, for example acetic acid, such as saturated or unsaturated dicarboxylic acids, for example oxalic, succinic, maleic or fumaric acid, such as hydroxy-carboxylic acids, for example glycolic, lactic, malic, tartaric or citric acid, such as amino acids, for example aspartic or glutamic acid, or with organic sulfonic acids, such as (C₁₋₄)alkyl-sulfonic acids (for example methanesulfonic acid) or arylsulfonic acids which are unsubstituted or substituted (for example by halogen). Preferred are salts formed with hydrochloric acid, methanesulfonic acid and maleic acid.

Prodrug derivatives of any compound of the present invention are derivatives of said compounds which following administration release the parent compound in vivo via some chemical or physiological process, e.g., a prodrug on being brought to the physiological pH or through enzyme action is converted to the parent compound. Exemplary prodrug derivatives are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art.

In view of the close relationship between the free compounds, the prodrug derivatives and the compounds in the form of their salts, whenever a compound is referred to in this context, a prodrug derivative and a corresponding salt is also intended, provided such is possible or appropriate under the circumstances.

The compounds, including their salts, can also be obtained in the form of their hydrates, or include other solvents used for their crystallization.

As described herein above, the compounds of the present invention are inhibitors of PTPases and, thus, may be employed for the treatment of conditions mediated by the PTPases. Accordingly, the compounds of formula (I) may be employed for treatment of insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer (such as prostate or breast cancer), osteoporosis, neurodegenerative and infectious diseases, and diseases involving inflammation and the immune system.

The present invention further provides pharmaceutical compositions comprising a therapeutically effective amount of a pharmacologically active compound of the instant invention, alone or in combination with one or more pharmaceutically acceptable carriers.

The pharmaceutical compositions according to the invention are those suitable for enteral, such as oral or rectal; transdermal and parenteral administration to mammals, including man, for the treatment of conditions mediated by PTPase activity, in particular, PTP-1B and TC PTP activity. Such conditions include insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer (such as prostate or breast cancer), osteoporosis, neurodegenerative and infectious diseases, and diseases involving inflammation and the immune system.

Thus, the pharmacologically active compounds of the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Accordingly, the present invention provides pharmaceutical compositions as described above for the treatment of conditions mediated by PTPases, preferably, insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer (such as prostate or breast cancer), osteoporosis, neurodegenerative and infectious diseases, and diseases involving inflammation and the immune system.

The pharmaceutical compositions may contain a therapeutically effective amount of a compound of the invention as defined above, either alone or in a combination with another therapeutic agent, e.g., each at an effective therapeutic dose as reported in the art. Such therapeutic agents include:

a) anti-diabetic agents, such as insulin, insulin derivatives and mimetics; insulin secretagogues such as the sulfonylureas, e.g., Glipizide, glyburide and Amaryl; insulinotropic sulfonylurea receptor ligands such as meglitinides, e.g., nateglinide and repaglinide; thiazolidone derivatives such as glitazones, e.g., pioglitazone and rosiglitazone, glucokinase activators; GSK3 (glycogen synthase kinase-3) inhibitors such as SB-517955, SB-4195052, SB-216763, NN-57-05441 and NN-57-05445; RXR ligands such as GW-0791 and AGN-194204; sodium-dependent glucose co-transporter inhibitors such as T-1095; glycogen phosphorylase A inhibitors such as BAY R3401; biguanides such as metformin; alpha-glucosidase inhibitors such as acarbose; GLP-1 (glucagon like peptide-1), GLP-1 analogs such as Exendin-4 and GLP-1 mimetics; modulators of PPARs (peroxisome proliferator-activated receptors), e.g., non-glitazone type PPARγ agonists such as N-(2-benzoylphenyl)-L-tyrosine analogues, e.g. GI-262570, and JTT501; DPPIV (dipeptidyl peptidase IV) inhibitors such as LAF237, MK-0431, saxagliptin and GSK23A; SCD-1 (stearoyl-CoA desaturase-1) inhibitors; DGAT1 and DGAT2 (diacylglycerol acyltransferase 1 and 2) inhibitors; ACC2 (acetyl CoA carboxylase 2) inhibitors; and breakers of AGE (advanced glycation end products);
b) anti-dyslipidemic agents such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin and rivastatin; HDL increasing compounds such as cholesterol ester transfer protein (CETP) inhibitors, e.g., JTT705; Apo-A1 analogs and mimetics; squalene synthase inhibitors; FXR (farnesoid X receptor) and LXR (liver X receptor) ligands; cholestyramine; fibrates; nicotinic acid; and aspirin;
c) anti-obesity agents such as phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine, ecopipam, ephedrine, and pseudoephedrine; cholesterol absorption modulators such as ZETIA® and KT6-971; and cannabinoid receptor antagonists such as rimonabant; and
d) anti-hypertensive agents, e.g., loop diuretics such as ethacrynic acid, furosemide and torsemide; angiotensin converting enzyme (ACE) inhibitors such as benazepril, captopril, enalapril, fosinopril, lisinopril, moexipril, perinodopril, quinapril, ramipril and trandolapril; inhibitors of the Na-K-ATPase membrane pump such as digoxin; neutralendopeptidase (NEP) inhibitors; ACE/NEP inhibitors such as omapatrilat, sampatrilat and fasidotril; angiotensin II antagonists such as candesartan, eprosartan, irbesartan, losartan, telmisartan and valsartan, in particular valsartan; renin inhibitors such as ditekiren, zankiren, terlakiren, aliskiren, RO 66-1132 and RO-66-1168; β-adrenergic receptor blockers such as acebutolol, atenolol, betaxolol, bisoprolol, metoprolol, nadolol, propranolol, sotalol and timolol; inotropic agents such as digoxin, dobutamine and milrinone; calcium channel blockers such as amlodipine, bepridil, diltiazem, felodipine, nicardipine, nimodipine, nifedipine, nisoldipine and verapamil; aldosterone receptor antagonists such as eplerenone; and aldosterone synthase inhibitors such as anastrazole and fadrazole.

Other specific anti-diabetic compounds are described by Patel Mona in Expert Opin Investig Drugs, 2003, 12(4), 623-633, in the FIGS. 1 to 7, which are herein incorporated by reference. A compound of the present invention may be administered either simultaneously, before or after the other active ingredient, either separately by the same or different route of administration or together in the same pharmaceutical formulation.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

Accordingly, the present invention provides pharmaceutical compositions comprising a therapeutically effective amount of a compound of the invention in combination with a therapeutically effective amount of another therapeutic agent, preferably selected from anti-diabetics, hypolipidemic agents, anti-obesity agents or anti-hypertensive agents, most preferably from antidiabetics or anti-obesity agents as described above.

The present invention further relates to pharmaceutical compositions as described above for use as a medicament.

The present invention further relates to use of pharmaceutical compositions or combinations as described above for the preparation of a medicament for the treatment of conditions mediated by PTPase activity, in particular, PTP-1B and TC PTP activity. Such conditions include insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer (such as prostate or breast cancer), osteoporosis, neurodegenerative and infectious diseases, and diseases involving inflammation and the immune system.

Thus, the present invention also relates to a compound of formula (I) for use as a medicament, to the use of a compound of formula (I) for the preparation of a pharmaceutical composition for treatment of conditions mediated by PTPase activity, in particular, PTP-1B and TC PTP activity, and to a pharmaceutical composition for use in conditions mediated by PTPase activity, in particular, PTP-1B and TC PTP activity, comprising a compound of formula (I), or a pharmaceutically acceptable salt thereof, in association with a pharmaceutically acceptable diluent or carrier therefore.

The present invention further provides a method for the treatment of conditions mediated by PTPase activity, in particular, PTP-1B and TC PTP activity, which method comprises administering a therapeutically effective amount of a compound of the present invention. A unit dosage for a mammal of about 50 to 70 kg may contain between about 1 mg and 1000 mg, advantageously between about 5 mg to 500 mg of the active ingredient. The therapeutically effective dosage of a compound of formula I is dependent on the species of warm-blooded animal (mammal), the body weight, age and individual condition, on the form of administration, and on the compound involved.

In accordance with the foregoing the present invention also provides a therapeutic combination, e.g., a kit, kit of parts, e.g., for use in any method as defined herein, comprising a compound of formula (I), or a pharmaceutically acceptable salt thereof, to be used concomitantly or in sequence with at least one pharmaceutical composition comprising at least another therapeutic agent, preferably selected from anti-diabetic agents, hypolipidemic agents, anti-obesity agents or anti-hypertensive agents. The kit may comprise instructions for its administration.

Similarly, the present invention provides a kit of parts comprising: (i) a pharmaceutical composition of the invention; and (ii) a pharmaceutical composition comprising a compound selected from an anti-diabetic, a hypolipidemic agent, an anti-obesity agent, an anti-hypertensive agent, or a pharmaceutically acceptable salt thereof, in the form of two separate units of the components (i) to (ii).

Likewise, the present invention provides a method as defined above comprising co-administration, e.g., concomitantly or in sequence, of a therapeutically effective amount of a compound of formula (I), or a pharmaceutically acceptable salt thereof, and a second drug substance, said second drug substance being an anti-diabetic, a hypolipidemic agent, an anti-obesity agent or an anti-hypertensive agent, e.g., as indicated above.

Preferably, a compound of the invention is administered to a mammal in need thereof.

Preferably, a compound of the invention is used for the treatment of a disease which responds to modulation of PTPase activity, in particular, PTP-1B and TC PTP activity.

Preferably, the condition associated with PTPase activity, in particular, PTP-1B and TC PTP activity, is selected from insulin resistance, glucose intolerance, obesity, diabetes mellitus, hypertension and ischemic diseases of the large and small blood vessels, conditions accompanying type 2 diabetes including dyslipidemia, e.g., hyperlipidemia and hypertriglyceridemia, atherosclerosis, vascular restenosis, irritable bowel syndrome, pancreatitis, adipose cell tumors and carcinomas such as liposarcoma, dyslipidemia, and other disorders where insulin resistance is indicated. In addition, the compounds of the present invention may be employed to treat cancer (such as prostate or breast cancer), osteoporosis, neurodegenerative and infectious diseases, and diseases involving inflammation and the immune system.

Finally, the present invention provides a method or use which comprises administering a compound of formula (I) in combination with a therapeutically effective amount of an anti-diabetic agent, a hypolipidemic agent, an anti-obesity agent or an anti-hypertensive agent.

Ultimately, the present invention provides a method or use which comprises administering a compound of formula (I) in the form of a pharmaceutical composition as described herein.

As used throughout the specification and in the claims, the term “treatment” embraces all the different forms or modes of treatment as known to those of the pertinent art and in particular includes preventive, curative, delay of progression and palliative treatment.

The above-cited properties are demonstrable in vitro and in vivo tests, using advantageously mammals, e.g., mice, rats, dogs, monkeys or isolated organs, tissues and preparations thereof. Said compounds can be applied in vitro in the form of solutions, e.g. preferably aqueous solutions, and in vivo either enterally, parenterally, advantageously intravenously, e.g. as a suspension or in aqueous solution. The dosage in vitro may range between about 10⁻³ molar and 10⁻¹¹ molar concentrations or between about 10⁻³ molar and 10⁻¹⁰ molar concentrations. A therapeutically effective amount in vivo may range depending on the route of administration, between about 0.1 and 500 mg/kg or between about 1 and 500 mg/kg, preferably between about 5 and 100 mg/kg.

The activity of a compound according to the invention may be assessed by the following methods or by following methods well described in the art (e.g. Peters G. et al. J. Biol. Chem., 2000, 275, 18201-09).

For example, the PTP-1B inhibitory activity in vitro may be determined as follows:

Assessment of human PTP-1B (hPTP-1B) activity in the presence of various agents is determined by measuring the amount of inorganic phosphate released from a phosphopeptide substrate using a 96-well microtiter plate format. The assay (100 μL) is performed in an assay buffer comprised of 50 mM TRIS (pH 7.5), 50 mM NaCl, 3 mM DTT at ambient temperature. The assay is typically performed in the presence of 0.4% dimethyl sulfoxide (DMSO). However, concentrations as high as 10% are used with certain poorly soluble compounds. A typical reaction is initiated by the addition of 0.4 pmoles of hPTP-1B (amino acids 1-411) to wells containing assay buffer, 3 nmoles of the synthetic phosphopeptide substrate (GNGDpYMPMSPKS), and the test compound. After 10 min, 180 μL malachite green reagent (0.88 mM malachite green, 8.2 mM ammonium molybdate, aqueous 1 N HCl, and 0.01% Triton X-100) is added to terminate the reaction. Inorganic phosphate, a product of the enzyme reaction, is quantitiated after 15 min as the green color resulting from complexing with the Malichite reagent and is determined as an A₆₂₀ using a Molecular Devices (Sunnyvale, Calif.) SpectraMAX Plus spectrophotometer. Test compounds are solubilized in 100% DMSO (Sigma, D-8779) and diluted in DMSO. Activity is defined as the net change in absorbance resulting from the activity of the uninhibited hPTP-1B_{[1-411]} minus that of a tube with acid-inactivated hPTP-1B_{[1-411]}.

The hPTP-1B_{[1-411]} is cloned by PCR from a human hippocampal cDNA library (Clonetech) and inserted into a pET 19-b vector (Novagen) at the Nco1 restriction site. E. coli strain BL21 (DE3) is transformed with this clone and stored as a stock culture in 20% glycerol at −80° C. For enzyme production, a stock culture is inoculated into Lb/Amp and grown at 37° C. Expression of PTP-1B is initiated by induction with 1 mM IPTG after the culture had reached an OD₆₀₀=0.6. After 4 h, the bacterial pellet is collected by centrifugation. Cells are resuspended in 70 mL lysis buffer (50 mM Tris, 100 mM NaCl, 5 mM DTT, 0.1% Triton X-100, pH7.6), incubated on ice for 30 min then sonicated (4×10 sec bursts at full power). The lysate is centrifuged at 100,000×g for 60 min and the supernatant is buffer exchanged and purified on a cation exchange POROS 20SP column followed by an anion exchange Source 30Q (Pharmacia) column, using linear NaCl gradient elutions. Enzyme is pooled, adjusted to 1 mg/mL and frozen at −80° C.

Alternatively, the assessment of human PTP-1B activity in the presence of various agents may be determined by measuring the hydrolysis products of known competing substrates. For example, cleavage of substrate para-nitrophenylphosphate (pNPP) results in the release of the yellow-colored para-nitrophenol (pNP) which can be monitored in real time using a spectrophotometer. Likewise, the hydrolysis of the fluorogenic substrate 6,8-difluoro-4-methylumbelliferyl phosphate ammonium salt (DiFMUP) results in the release of the fluorescent DiFMU which can be readily followed in a continuous mode with a fluorescence reader (Anal. Biochem. 273, 41, 1999; Anal. Biochem. 338, 32, 2005):

pNPP Assay

Compounds were incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 for 5 min at room temperature. The reaction is initiated by the addition of pNPP (2 mM final concentration) and run for 120 min at room temperature. Reactions are quenched with 5 N NaOH. Absorbance at 405 nm is measured using any standard 384 well plate reader.

### DiFMUP Assay

Compounds are incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 (or 0.001% BSA) for 5 min at room temperature. The reaction is initiated by the addition of DiFMUP (6 μM final concentration) and run kinetically on fluorescence plate reader at 355 nm excitation and 460 nm emission wavelengths. Reaction rates over 15 min are used to calculate inhibition.

PTP-1B_{[1-298]} is expressed in E. coli BL21 (DE3) containing plasmids constructed using pET19b vectors (Novagen). The bacteria is grown in minimal media using an “On Demand” Fed-batch strategy. Typically, a 5.5 liter fermentation is initiated in Fed-batch mode and grown overnight unattended at 37° C. Optical densities varied between 20-24 OD₆₀₀ and the cultures are induced at 30° C. with IPTG to a final concentration of 0.5 mM. The bacterial cells are harvested 8 hours later and yield 200-350 gm (wet weight). The cells are frozen as pellets and stored at −80° C. until use. All steps are performed at 4° C. unless noted. Cells (˜15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication (4×10 second burst, full power) using a Virsonic 60 (Virtus). The pellet is collected at 35,000×g, resuspended in 25 mL of lysis buffer using a Polytron and collected as before. The two supernatants are combined and centrifuged for 30 min at 100,000×g. The soluble lysate could be stored at this stage at −80° C. or used for further purification. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. An analytical column (4.6×100 mm) is run in a similar fashion except the flow rate was reduced to 10 mL/min. Protein is eluted from the column using a linear salt gradient (75-500 mM NaCl in 25 CV). Fractions containing PTP-1B_{[1-298]} are identified and pooled according to SDS-PAGE analyses. Final purification is performed using Sephacryl S-100 HR (Pharmacia). The column (2.6×35 cm) is equilibrated with 50 mM HEPES, 100 mM NaCl, 3 mM DTT, pH 7.5 and run at a flow rate of 2 mL/min. The final protein is pooled and concentrated to ˜5 mg/mL using an Ultrafree-15 concentrator (Millipore) with a MWCO 10,000. The concentrated protein is stored at −80° C. until use.

Competitive binding to the active site of the enzyme may be determined as follows:

Ligand binding is detected by acquiring ¹H-¹⁵N HSQC spectra on 250 μL of 0.15 mM PTP-1B_{[298]} in the presence and absence of added compound (1-2 mM). The binding is determined by the observation of ¹⁵N- or ¹H-amide chemical shift changes in two dimensional HSQC spectra upon the addition of a compound to ¹⁵N-label protein. Because of the ¹⁵N spectral editing, no signal from the ligand is observed, only protein signals. Thus, binding can be detected at high compound concentrations. Compounds which caused a pattern of chemical shift changes similar to the changes seen with known active site binders are considered positive.

All proteins are expressed in E. coli BL21 (DE3) containing plasmids constructed using pET19b vectors (Novagen). Uniformly ¹⁵N-labeled PTP-1B_{[1-298]} is produced by growth of bacteria on minimal media containing ¹⁵N-labeled ammonium chloride. All purification steps are performed at 4° C. Cells (˜15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication. The pellet is collected at 35,000×g, resuspended in 25 mL of lysis buffer using a Polytron and collected as before. The two supernatants are combined and centrifuged for 30 min at 100,000×g. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. Protein is eluted from the column using a linear salt gradient (75-500 mM NaCl in 25 CV). Fractions containing PTP-1B's are identified and pooled according to SDS-PAGE analyses. PTP-1B_{[1-298]} is further purified by anion exchange chromatography using a POROS 20 HQ column (1×10 cm). The pool from cation exchange chromatography is concentrated and buffer exchanged in 50 mM Tris-HCl, pH 7.5 containing 75 mM NaCl and 5 mM DTT. Protein is loaded onto column at 20 mL/min and eluted using a linear NaCl gradient (75-500 mM in 25 CV). Final purification is performed using Sephacryl S-100 HR (Pharmacia) (50 mM HEPES, 100 mM NaCl, 3 mM DTT, pH 7.5). The NMR samples are composed of uniformly ¹⁵N-labeled PTP-1B₁₋₂₉₈ (0.15 mM) and inhibitor (1-2 mM) in a 10% D₂O/90% H₂O Bis-Tris-d₁₉ buffer (50 mM, pH=6.5) solution containing NaCl (50 mM), DL-1,4-Dithiothreitol-d₁₀ (5 mM) and Sodium azide (0.02%).

The ¹H-¹⁵N HSQC NMR spectra are recorded at 20° C., on Bruker DRX500 or DMX600 NMR spectrometers. In all NMR experiments, pulsed field gradients are applied to afford the suppression of solvent signal. Quadrature detection in the indirectly detected dimensions is accomplished by using the States-TPPI method. The data are processed using Bruker software and analyzed using NMRCompass software (MSI) on Silicon Graphics computers.

The glucose and insulin lowering activity in vivo may be evaluated as follows:

Adult male C57BL ob/ob mice (Jackson Lab, Bar Harbor, Me.) at the age of 11 weeks are housed six per cage in a reversed light cycle room (light on from 6:00 p.m. to 6:00 a.m.) and given access to Purina rodent chow and water ad libitum. On day 1 tail blood samples are taken at 8:00 am and plasma glucose levels are determined. The animals are randomly assigned to the control and compound groups. The means of plasma glucose values of the groups are matched. Animals are then orally dosed with vehicle (0.5% carboxymethyl-cellulose with 0.2% Tween-80) or compounds (at 30 mg/kg) in vehicle. The mice are dosed daily for a total of 3 days. On day 4 basal blood samples are taken. The plasma samples are analyzed for glucose concentrations using a YSI2700 Dual Channel Biochemistry Analyzer (Yellow Springs Instrument Co., Yellow Springs, Ohio) and insulin concentrations using an ELISA assay.

The following Examples are intended to illustrate the invention and are not to be construed as being limitations thereon. Temperatures are given in degrees Centrigrade (° C.). If not mentioned otherwise, all evaporations are performed under reduced pressure, preferably between about 15 and 100 mmHg (=20-133 mbar). The structure of final products, intermediates and starting materials is confirmed by standard analytical methods, e.g. microanalysis, melting point (mp) and spectroscopic characteristics (e.g. MS, IR, NMR). In general, abbreviations used are those conventional in the art.

Method A: 4.6 mm×5 cm C-8 reverse phase column, 3 μm particle size running a gradient of 10-90% MeCN/water (5 mM ammonium bicarbonate) over a period of 2 min at a flow rate of 4 mL/min at 50° C. (3 μL injection). DAD-UV detection, 220-600 nm.

### EXAMPLE 1

### 5-[2-Hydroxy-5-(1H-pyrrol-2-yl)-phenyl]-1,1-dioxo-1,2,5-thiadiazolidin-3-one

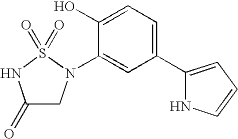

### A. 1-Benzyloxy-4-bromo-2-nitrobenzene

A solution of 4-bromo-2-nitrophenol (226.81 g, 1.04 mol) in DMF (2 L) is treated with potassium carbonate (172.55 g, 1.24 mol). The suspension is agitated by mechanical stirring and heated to 50° C. Benzyl bromide (148 mL, 1.25 mol) is added and the suspension is heated to 62° C. for 3 h and 72° C. for an additional 40 min., at which point the reaction is judged complete by LCMS. The suspension is filtered and the filter-cake is washed in portions with DMF (0.5 L). Water (5 L) is added to the DMF solution, which is then cooled gradually to 23° C. with vigorous stirring. The precipitate is filtered and dried in a vacuum oven to afford 1-benzyloxy-4-bromo-2-nitrobenzene as a yellow solid: ¹H NMR (CDCl₃) δ 7.89 (d, J=2 Hz, 1H), 7.50 (dd, J=12, 2 Hz, 1H), 7.30 (m, 5H), 6.93 (d, J=12 Hz, 1H), 5.14 (s, 2H).

### B. 2-Benzyloxy-5-bromophenylamine

To a solution of 1-benzyloxy-4-bromo-2-nitrobenzene (10.6 g, 34.4 mmol) in EtOH (70 mL) and AcOH (26 mL), is added iron powder (9.61 g, 172 mmol). The suspension is agitated by mechanical stirring and heated at 100° C. for 2 h, at which point the reaction is judged complete by LCMS. The EtOH and AcOH are removed in vacuo. DCM (250 mL) and water (250 mL) are added and the suspension is stirred vigorously with a mechanical stirrer. Heating is continued for 4 h and the reaction is judged complete by LCMS. The suspension is filtered through Celite and the solid is washed with DCM. The filtrate is washed with water (250 mL), extracted with DCM, dried over Na₂SO₄, filtered and concentrated under reduced pressure to afford 2-benzyloxy-5-bromophenylamine: ¹H NMR (CDCl₃) δ 7.39-7.35 (m, 5H), 6.87 (d, J=4 Hz, 1H), 6.82 (dd, J=8.0, 4.0 Hz, 1H), 6.72 (d, J=8 Hz, 1H), 5.08 (s, 2H), 3.89 (br s, 2H); MS (M+1)⁺=278, 280.

### C. (2-Benzyloxy-5-bromophenylamino)-acetic acid ethyl ester

A solution of 2-benzyloxy-5-bromophenylamine (138.89 g, 0.499 mol) in acetonitrile (2 L), AcOH (1 L) and ethyl glyoxalate (153 mL, 0.749 mol) is cooled to 11° C. and sodium triacetoxyborohydride (211.6 g, 0.998 mol) is added as a suspension. The suspension is stirred for 5 min., at which point the reaction is judged complete by LCMS. The AcOH and acetonitrile are removed in vacuo. The solid is dissolved in DCM and washed with saturated sodium bicarbonate. The organic layer is washed with saturated NaCl, dried over Na₂SO₄ and filtered through a pad of silica gel. The product is eluted with 1 L portions of DCM. Removal of DCM in vacuo affords (2-benzyloxy-5-bromophenylamino)-acetic acid ethyl ester: ¹H NMR (CDCl₃) δ 7.33 (m, 5H), 6.78 (dd, J=8.0, 4.0 Hz, 1H), 6.68 (d, J=8 Hz, 1H), 6.63 (d, J=4.0 Hz, 1H), 5.08 (s, 2H), 4.25 (q, J=8.0 Hz, 2H), 3.90 (s, 2H), 1.29 (t, J=8 Hz, 3H); (M+H)⁺=364, 366.

### D. 2-Benzyloxy-5-bromophenyl-N-(t-butoxycarbonylsulfamoyl)-acetic acid ethyl ester

Methylene chloride (250 mL) is cooled to 0° C. Chlorosulfonyl isocyanate (23.97 mL, 0.27 mol) is added, followed by 2-methyl-2-propanol (28.7 mL, 0.30 mol), and the solution is stirred for 30 min. A solution of 2-benzyloxy-5-bromophenylamino-acetic acid ethyl ester (91.20 g, 0.25 mol) and triethylamine (38.4 mL, 0.275 mol) in DCM (250 mL) is added rapidly dropwise via addition funnel. The solution is stirred for 5 min., at which point the reaction is judged complete by LCMS. The DCM is removed in vacuo. The solid is dissolved in EtOAc and washed with 1N HCl solution. The organic layer is washed with saturated sodium chloride, dried over Na₂SO₄, filtered and concentrated in vacuo to afford 2-benzyloxy-5-bromophenyl-N-(t-butoxycarbonylsulfamoyl)-acetic acid ethyl ester: ¹H NMR (CDCl₃) δ 7.72 (d, J=4 Hz, 1H), 7.47 (s, 1H), 7.31-7.27 (m, 6H), 6.75 (d, J=12 Hz, 1H), 5.08 (s, 2H), 4.44 (s, 2H), 4.08 (q, J=8 Hz, 2H), 1.36 (s, 9H), 1.17 (t, J=8 Hz, 3H); MS (M−1)⁻=541,543.

### E. 5-(2-Benzyloxy-5-bromophenyl)-1,1-dioxo-1,2,5-thiadiazolidin-3-one

To a solution of 2-benzyloxy-5-bromophenyl-N-(t-butoxycarbonylsulfamoyl)-acetic acid ethyl ester (114.81 g, 0.211 mol) in DCM (560 mL) is added TFA (280 mL). The reaction is stirred for 5 min., then concentrated in vacuo. The resulting solid is dissolved in THF (2 L), and the solution is cooled to 0° C. A solution of potassium tert-butoxide in THF (1 M) is added dropwise in portions until the reaction is judged complete by LCMS. Aqueous HCl (350 mL, 0.350 mol) is added and the THF is removed in vacuo. NaCl is added until the aqueous phase is saturated, at which point it is extracted with EtOAc (1 L). The organic layer is extracted with saturated NaCl, dried over Na₂SO₄, filtered and concentrated in vacuo to afford crude 5-(2-benzyloxy-5-bromophenyl)-1,1-dioxo-1,2,5-thiadiazolidin-3-one. Recrystallization from acetonitrile/water (1:1) affords pure 5-(2-benzyloxy-5-bromophenyl)-1,1-dioxo-1,2,5-thiadiazolidin-3-one: MS (M−1)⁻=395, 397.

### F. 2-[4-Benzyloxy-3-(1,1,4-trioxo-1,2,5-thiadiazolidin-2-yl)-phenyl]-pyrrole-1-carboxylic acid tert-butyl ester

To a stirred solution 5-(2-benzyloxy-5-bromophenyl)-1,1-dioxo-1,2,5-thiadiazolidin-3-one (50 mg, 0.126 mmol) in DME (2 mL) is added Pd(PPh₃)₄ (15 mg, 0.013 mmol), pyrrole-2-boronic acid-1-carboxylic acid tert-butyl ester (53 mg, 0.252 mmol) and 250 μL of a 2M Na₂CO₃ solution. The solution is heated at 80° C. for 18 h. LC/MS of the reaction mixture reveals approximately 90% consumption of the starting aryl bromide, so the mixture is diluted with EtOAc and 1N HCl. The organic layer is separated and concentrated in vacuo to afford a dark brown oil, which is purified using reverse phase chromatography to afford 2-[4-benzyloxy-3-(1,1,4-trioxo-1,2,5-thiadiazolidin-2-yl)-phenyl]-pyrrole-1-carboxylic acid tert-butyl ester, which is used directly in the next step: MS (M−1)⁻=482.

### G. 2-[4-Hydroxy-3-(1,1,4-trioxo-1,2,5-thiadiazolidin-2-yl)-phenyl]-pyrrole-1-carboxylic acid tert-butyl ester

To a mixture of Pd/C (5 mg) in EtOH (5 mL) is added 2-[4-benzyloxy-3-(1,1,4-trioxo-1,2,5-thiadiazolidin-2-yl)-phenyl]-pyrrole-1-carboxylic acid tert-butyl ester (20 mg, 0.041 mmol) in EtOAc (5 mL). The flask is placed under an atmosphere of H₂ for 18 h, at which time LCMS of the reaction mixture reveals complete deprotection of the benzyl group, as well as some undesired pyrrole reduction to the corresponding pyrrolidine. The crude mixture is purified via prep HPLC to afford 2-[4-hydroxy-3-(1,1,4-trioxo-1,2,5-thiadiazolidin-2-yl)-phenyl]-pyrrole-1-carboxylic acid tert-butyl ester, which is directly used in the next step: MS (M−1)⁻=392.

### H. 5-[2-Hydroxy-5-(1H-pyrrol-2-yl)-phenyl]-1,1-dioxo-1,2,5-thiadiazolidin-3-one

To 2-[4-hydroxy-3-(1,1,4-trioxo-1,2,5-thiadiazolidin-2-yl)-phenyl]-pyrrole-1-carboxylic acid tert-butyl ester (5 mg, 0.013 mmol) in DCM (15 mL) is added 1 drop of TFA. The reaction is stirred for 1 h. Concentration in vacuo, followed by prep HPLC purification affords 5-[2-hydroxy-5-(1H-pyrrol-2-yl)-phenyl]-1,1-dioxo-1,2,5-thiadiazolidin-3-one as a clear film: MS (M−1)⁻=292.

### EXAMPLE 2

The following compounds are prepared using appropriate starting materials and general methods described in Example 1, with the exception that Step H is eliminated. Example 2-2 is prepared using benzyloxymethylpyrazoleboronic acid (Tet Lett, 1993, 34, 8237).

| | | | Retention |
|---|---|---|---|
| | | | time (min) |
| Example | Chemical Name | MS (m/z) | Method |
| 2-1 | 5-(4-Hydroxybiphenyl-3-yl)-1,1-dioxo-1,2,5- | (M − 1)⁻ = 303 | |
| | thiadiazolidin-3-one | | |
| 2-2 | 5-[2-Hydroxy-5-(2H-pyrazol-3-yl)-phenyl]-1,1- | (M − 1)⁻ = 293 | |
| | dioxo-1,2,5-thiadiazolidin-3-one | | |
| 2-3 | 5-[2-Hydroxy-5-(1-methyl-1H-pyrazol-4-yl)- | (M − 1)⁻ = 307 | |
| | phenyl]-1,1-dioxo-1,2,5-thiadiazolidin-3-one | | |
| 2-4 | 5-(5-Furan-3-yl-2-hydroxyphenyl)-1,1-dioxo- | (M − 1)⁻ = 293 | |
| | 1,2,5-thiadiazolidin-3-one | | |
| 2-5 | 5-[2-Hydroxy-5-(1H-pyrazol-4-yl)-phenyl]-1,1- | (M − 1)⁻ = 293 | |
| | dioxo-1,2,5-thiadiazolidin-3-one | | |
| 2-6 | 5-(4′-Acetyl-4-hydroxybiphenyl-3-yl)-1,1-dioxo- | (M − 1)⁻ = 345 | |
| | 1,2,5-thiadiazolidin-3-one | | |
| 2-7 | 5-(4′-Benzoyl-4-hydroxybiphenyl-3-yl)-1,1- | (M − 1)⁻ = 407 | 1.21 A |
| | dioxo-1,2,5-thiadiazolidin-3-one | | |

### EXAMPLE 3

### 5-[2-Hydroxy-5-(1H-pyrrol-3-yl)-phenyl]-1,1-dioxo-1,2,5-thiadiazolidin-3-one

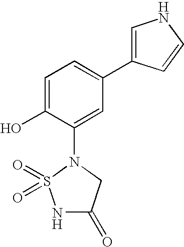

### A. 5-[2-Hydroxy-5-(1-triisopropylsilanyl-1H-pyrrol-3-yl)-phenyl]-1,1-dioxo-1,2,5-thiadiazolidin-3-one

The title compound is prepared analogously to Example 1 using 1-triisopropylsilanylpyrrol-3-boronic acid with the exception that Step H is eliminated.

### B. 5-[2-Hydroxy-5-(1H-pyrrol-3-yl)-phenyl]-1,1-dioxo-1,2,5-thiadiazolidin-3-one

To a solution of 5-[2-hydroxy-5-(1-triisopropylsilanyl-1H-pyrrol-3-yl)-phenyl]-1,1-dioxo-1,2,5-thiadiazolidin-3-one (20 mg, 0.04 mmol) in CH₃CN (2 mL) is added HF-pyridine (50%, 0.048 mL, 0.1 mmol) and the mixture is stirred at ambient temperature for 1.5 h. The mixture is concentrated and purified by RP chromatography with ammonium formate to give the title compound: Retention time=0.63 min (Method A); MS (M−1)⁻=292.

### EXAMPLE 4

### Methanesulfonic acid 4′-hydroxy-3′-(1,1,4-trioxo-1,2,5-thiadiazolidin-2-yl)-biphenyl-3-yl ester

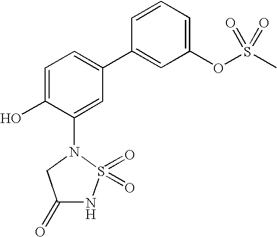

### A. Methanesulfonic acid 3-(4,4,5,5-tetramethyl-[1,3,2]dioxaborolan-2-yl)-phenyl ester

To a stirring solution of 3-(4,4,5,5-tetramethyl-[1,3,2]dioxaborolan-2-yl)-phenol (0.050 g, 0.227 mmol) in DCM (10 mL) and triethylamine (0.064 mL, 0.449 mmol) is added MsCl (0.035 mL, 0.452 mmol) dropwise at 0° C. The reaction mixture is stirred for 2.5 h. The mixture is poured into 1N HCl and extracted with DCM (3×15 mL). The organic layers are combined and concentrated to afford methanesulfonic acid 3-(4,4,5,5-tetramethyl-[1,3,2]dioxaborolan-2-yl)-phenyl ester: (M+NH₄)=316.

### B. Methanesulfonic acid 4′-benzyloxy-3′-(1,1,4-trioxo-1,2,5-thiadiazolidin-2-yl)-biphenyl-3-yl ester

In a microwave vial is added methanesulfonic acid 3-(4,4,5,5-tetramethyl-[1,3,2]dioxaborolan-2-yl)-phenyl ester (0.060 g, 0.201 mmol), 5-(2-benzyloxy-5-bromophenyl)-1,1-dioxo-1,2,5-thiadiazolidin-3-one (0.04 g, 0.101 mmol), Pd(PPh₃)₄ (0.029 g, 0.025 mmol) and 2 M Na₂CO₃ (0.125 mL). DME is added and the vial is capped and placed in the microwave for a total of 30 min. at 110° C. The reaction mixture is filtered through Celite and washed with MeOH. The filtrate is concentrated and the residue is purified via Biotage Sp1, eluting with 5-65% EtOH/H₂O to afford methanesulfonic acid 4′-benzyloxy-3′-(1,1,4-trioxo-1,2,5-thiadiazolidin-2-yl)-biphenyl-3-yl ester: MS (M−1)=487.2.

### C. Methanesulfonic acid 4′-hydroxy-3′-(1,1,4-trioxo-1,2,5-thiadiazolidin-2-yl)-biphenyl-3-yl ester

To a stirring solution of methanesulfonic acid 4′-benzyloxy-3′-(1,1,4-trioxo-1,2,5-thiadiazolidin-2-yl)-biphenyl-3-yl ester (0.010 g, 0.020 mmol) in EtOH/EtOAc (1:3, 10 mL) is added 5% Pd/C (0.005 g). The mixture is stirred under an atmosphere of H₂ of 1.5 h. The reaction mixture is filtered over Celite, washed with EtOH and concentrated. The residue is purified via prep HPLC to afford methanesulfonic acid 4′-hydroxy-3′-(1,1,4-trioxo-1,2,5-thiadiazolidin-2-yl)-biphenyl-3-yl ester: MS (M−1)=397.

### EXAMPLE 5

### 5-(3′-Amino-4-hydroxybiphenyl-3-yl)-1,1-dioxo-1,2,5-thiadiazolidin-3-one

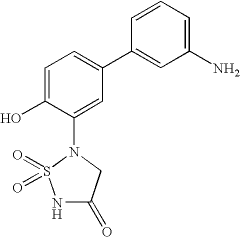

### A. 5-(3′-Amino-4-benzyloxybiphenyl-3-yl)-1,1-dioxo-1,2,5-thiadiazolidin-3-one

To a 20 mL vial containing 5-(2-benzyloxy-5-bromophenyl)-1,1-dioxo-1,2,5-thiadiazolidin-3-one (Example 1, Step E) (1.00 g, 2.52 mmol), 3-(4,4,5,5-tetramethyl-1,3,2-dioxaborolan-2-yl)-phenylamine (690 mg, 5.04 mmol) and Pd(PPh₃)₄ (291 mg, 0.252 mmol) is added DME (12 mL). The solution is separated into 4 microwave vessels and to each of these vessels is added a solution of 2 M Na₂CO₃ (1.25 mL). The reaction mixtures are subjected to microwave irradiation at 110° C. for 45 min. The contents of the 4 vessels are combined, concentrated in vacuo and purified using reverse phase chromatography to afford 5-(3′-amino-4-benzyloxybiphenyl-3-yl)-1,1-dioxo-1,2,5-thiadiazolidin-3-one, which is immediately used in the following step without removal of the EtOH/water eluent: MS (M−1)⁻=408.

### B. 5-(3′-Amino-4-hydroxybiphenyl-3-yl)-1,1-dioxo-1,2,5-thiadiazolidin-3-one

To 5-(3′-amino-4-benzyloxybiphenyl-3-yl)-1,1-dioxo-1,2,5-thiadiazolidin-3-one in EtOH/water is added a mixture of Pd/C (100 mg) in EtOH (10 mL). The flask is placed under an atmosphere of H₂ for 48 h. Removal of the Pd/C by filtration through a pad of Celite, followed by concentration in vacuo and purification by reverse phase chromatography affords 5-(3′-amino-4-hydroxybiphenyl-3-yl)-1,1-dioxo-1,2,5-thiadiazolidin-3-one as a light brown solid: Retention time=0.63 min (Method A); MS (M−1)⁻=318.

### EXAMPLE 6

The following compounds are prepared using appropriate starting materials and general methods described in Example 5. Example 6-17 requires the conversion of methyl ester to the ethyl ester prior to the debenzylation step. The debenzylation of Examples 6-18 through 6-22 are performed using Pd(OH)₂ and for Example 6-13 and 6-14, BBr₃ in DCM is used. Resin bound (PPh₃)₄ is used for Examples 6-13 to 6-22.

| | | | Retention |
|---|---|---|---|
| | | | time (min) |
| Example | Chemical Name | MS (m/z) | Method |
| 6-1 | 5-(4-Hydroxy-2′-methylbiphenyl-3-yl)-1,1- | (M − 1)⁻ = 317 | 1.02 A |
| | dioxo-1,2,5-thiadiazolidin-3-one | | |
| 6-2 | 5-[2-Hydroxy-5-(1H-indol-2-yl)-phenyl]-1,1- | (M − 1)⁻ = 342 | 1.19 A |
| | dioxo-1,2,5-thiadiazolidin-3-one | | |
| 6-3 | [4′-Hydroxy-3′-(1,1,4-trioxo-1,2,5- | (M − 1)⁻ = 342 | |
| | thiadiazolidin-2-yl)-biphenyl-3-yl]-acetonitrile | | |
| 6-4 | 4′-Hydroxy-3′-(1,1,4-trioxo-1,2,5- | (M − 1)⁻ = 399 | |
| | thiadiazolidin-2-yl)-biphenyl-3-carboxylic acid | | |
| | (2-cyanoethyl)-amide | | |
| 6-5 | 3-[4′-Hydroxy-3′-(1,1,4-trioxo-1,2,5- | (M − 1)⁻ = 389 | |
| | thiadiazolidin-2-yl)-biphenyl-3-yl]-propionic | | |
| | acid methyl ester | | |
| 6-6 | 4′-Hydroxy-3′-(1,1,4-trioxo-1,2,5- | (M − 1)⁻ = 417 | |
| | thiadiazolidin-2-yl)-biphenyl-3-carboxylic acid | | |
| | (2-carbamoylethy)-amide | | |
| 6-7 | 5-[3′-(2-Aminoethyl)-4-hydroxybiphenyl-3-yl]- | (M − 1)⁻ = 347 | |
| | 1,1-dioxo-1,2,5-thiadiazolidin-3-one | | |
| 6-8 | 5-(3′-Aminomethyl-4-hydroxybiphenyl-3-yl)- | (M − 1)⁻ = 332 | |
| | 1,1-dioxo-1,2,5-thiadiazolidin-3-one | | |
| 6-9 | 5-(2-Hydroxy-5-pyridin-3-yl-phenyl)-1,1- | (M − 1)⁻ = 304 | 0.69 A |
| | dioxo-1,2,5-thiadiazolidin-3-one | | |
| 6-10 | 5-(4-Hydroxy-2′-methoxy-biphenyl-3-yl)-1,1- | (M − 1)⁻ = 333 | 0.98 A |
| | dioxo-1,2,5-thiadiazolidin-3-one | | |
| 6-11 | 5-(2-Hydroxy-5-pyridin-4-yl-phenyl)-1,1- | (M − 1)⁻ = 304 | 0.61 A |
| | dioxo-1,2,5-thiadiazolidin-3-one | | |
| 6-12 | [4′-Hydroxy-3′-(1,1,4-trioxo-1,2,5- | (M − 1)⁻ = 361 | 0.54 A |
| | thiadiazolidin-2-yl)-biphenyl-4-yl]-acetic acid | | |
| 6-13 | 5-(4′-Chloro-4-hydroxybiphenyl-3-yl)-1,1- | (M − 1)⁻ = 337 | 1.15 A |
| | dioxo-1,2,5-thiadiazolidin-3-one | | |
| 6-14 | 5-(3′-Chloro-4-hydroxybiphenyl-3-yl)-1,1- | (M − 1)⁻ = 337 | 1.13 A |
| | dioxo-1,2,5-thiadiazolidin-3-one | | |
| 6-15 | 5-[2-Hydroxy-5-(6-methoxypyridin-3-yl)- | (M − 1)⁻ = 334 | 0.84 A |
| | phenyl]-1,1-dioxo-1,2,5-thiadiazolidin-3-one | | |
| 6-16 | 5-[5-(6-Fluoropyridin-3-yl)-2-hydroxyphenyl]- | (M − 1)⁻ = 322 | 0.76 A |
| | 1,1-dioxo-1,2,5-thiadiazolidin-3-one | | |
| 6-17 | 3-[4′-Hydroxy-3′-(1,1,4-trioxo-1,2,5- | (M − 1)⁻ = 403 | 1.19 A |
| | thiadiazolidin-2-yl)-biphenyl-3-yl]-propionic | | |
| | acid ethyl ester | | |
| 6-18 | 5-(4-Hydroxy-3′-methylbiphenyl-3-yl)-1,1- | (M − 1)⁻ = 317 | |
| | dioxo-1,2,5-thiadiazolidin-3-one | | |
| 6-19 | 5-(3′-Fluoro-4-hydroxybiphenyl-3-yl)-1,1- | (M − 1)⁻ = 321 | 1.06 A |
| | dioxo-1,2,5-thiadiazolidin-3-one | | |
| 6-20 | 5-(4′-Fluoro-4-hydroxybiphenyl-3-yl-1,1- | (M − 1)⁻ = 321 | 1.02 A |
| | dioxo-1,2,5-thiadiazolidin-3-one | | |
| 6-21 | 5-(4-Hydroxy-4′-methylbiphenyl-3-yl)-1,1- | (M − 1)⁻ = 317 | 1.11 A |
| | dioxo-1,2,5-thiadiazolidin-3-one | | |
| 6-22 | 3-[4′-Hydroxy-3′-(1,1,4-trioxo-1,2,5- | (M − 1)⁻ = 356 | 1.03 A |
| | thiadiazolidin-2-yl)-biphenyl-3-yl]-propionitrile | | |
| 6-23 | 4′-Hydroxy-3′-(1,1,4-trioxo-1,2,5- | (M − 1)⁻ = 329 | 0.94 A |
| | thiadiazolidin-2-yl)-biphenyl-3-carbonitrile | | |
| 6-24 | 5-(4-Hydroxy-3′,5′-dimethylbiphenyl-3-yl)-1,1- | (M − 1)⁻ = 331 | 1.24 A |
| | dioxo-1,2,5-thiadiazolidin-3-one | | |
| 6-25 | 5-(4-Hydroxy-3′-methoxybiphenyl-3-yl)-1,1- | (M − 1)⁻ = 333 | |
| | dioxo-1,2,5-thiadiazolidin-3-one | | |

### EXAMPLE 7

### N-(2-Hydroxyethyl)-2-[4′-hydroxy-3′-(1,1,4-trioxo-1,2,5-thiadiazolidin-2-yl)-biphenyl-4-yl]-acetamide

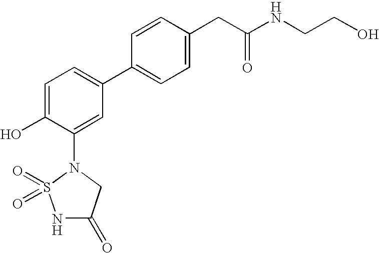

### A. 2-[4′-Benzyloxy-3′-(1,1,4-trioxo-1,2,5-thiadiazolidin-2-yl)-biphenyl-4-yl]-N-(2-hydroxyethyl)-acetamide

To a solution of [4′-benzyloxy-3′-(1,1,4-trioxo-1,2,5-thiadiazolidin-2-yl)-biphenyl-4-yl]-acetic acid (Example 6-12, prior to hydrogenation) (100 mg, 0.221 mmol), in THF (10 mL) is added EDCI (51 mg, 0.265 mmol), HOBt (36 mg, 0.265 mmol) and ethanolamine (0.031 mL, 0.442 mmol). The reaction mixture is stirred for 18 h and diluted with H₂O/EtOAc. The organic layer is separated, washed with brine, dried over Na₂SO₄, filtered and evaporated to dryness. Preparative HPLC purification affords 2-[4′-benzyloxy-3′-(1,1,4-trioxo-1,2,5-thiadiazolidin-2-yl)-biphenyl-4-yl]-N-(2-hydroxyethyl)-acetamide, which is used directly in the next step: MS (M−1)⁻=494.

### B. N-(2-Hydroxyethyl)-2-[4′-hydroxy-3′-(1,1,4-trioxo-1,2,5-thiadiazolidin-2-yl) biphenyl-4-yl]-acetamide

Debenzylation is conducted according to Example 5, Step B: Retention time=0.64 min (Method A); MS (M−1)⁻=404.

### EXAMPLE 8

### 2,2,2-Trifluoro-N-[4′-hydroxy-3′-(1,1,4-trioxo-1,2,5-thiadiazolidin-2-yl)-biphenyl-3-yl]-acetamide

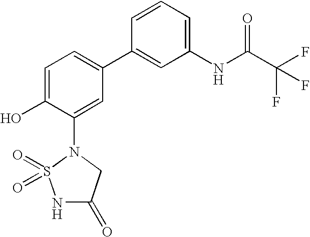

### A. N-[4′-Benzyloxy-3′-(1,1,4-trioxo-1,2,5-thiadiazolidin-2-yl)-biphenyl-3-yl]-2,2,2-trifluoroacetamide

To 5-(3′-amino-4-benzyloxybiphenyl-3-yl)-1,1-dioxo-1,2,5-thiadiazolidin-3-one (Example 5, Step A) (100 mg, 0.244 mmol), is added methyl trifluoromethylacetate (1 mL). The reaction mixture is heated to 60° C. and stirred for 2 h. Concentration of the reaction in vacuo affords N-[4′-benzyloxy-3′-(1,1,4-trioxo-1,2,5-thiadiazolidin-2-yl)-biphenyl-3-yl]-2,2,2-trifluoroacetamide to be used directly in the next step: MS (M−1)⁻=504.

### B. 2,2,2-Trifluoro-N-[4′-hydroxy-3′-(1,1,4-trioxo-1,2,5-thiadiazolidin-2-yl)-biphenyl-3-yl]-acetamide

Debenzylation is conducted according Example 5, Step B: Retention time=1.08 min (Method A); MS (M-1)⁻=414.

### EXAMPLE 9

### 1-Ethyl-3-[4′-hydroxy-3′-(1,1,4-trioxo-1,2,5-thiadiazolidin-2-yl)-biphenyl-3-yl]-urea

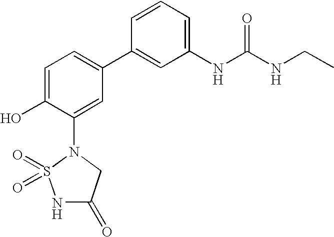

### A. 1-[4′-Benzyloxy-3′-(1,1,4-trioxo-1,2,5-thiadiazolidin-2-yl)-biphenyl-3-yl]-3-ethyl-urea

To a stirring solution of 5-(3′-amino-4-benzyloxybiphenyl-3-yl)-1,1-dioxo-1,2,5-thiadiazolidin-3-one (Example 5, Step A) (100 mg, 0.244 mmol), in DCE (10 mL) is added ethyl isocyanate (0.04 mL, 0.488 mmol). The reaction mixture is heated to 60° C. for 1 h. Concentration of the reaction mixture in vacuo affords 1-[4′-benzyloxy-3′-(1,1,4-trioxo-1,2,5-thiadiazolidin-2-yl)-biphenyl-3-yl]-3-ethylurea, to be used directly in the next step: MS (M−1)⁻=479.

### B. 1-Ethyl-3-[4′-hydroxy-3′-(1,1,4-trioxo-1,2,5-thiadiazolidin-2-yl)-biphenyl-3-yl]-urea

Debenzylation is conducted according to Example 5, Step B: Retention time=0.81 min (Method A); MS (M−1)⁻=389.

### EXAMPLE 10

The following compounds are prepared using the following: the general methods described in Examples 8 and 9, 3-aminomethylphenyl boronic acid or 3-(4,4,5,5-tetramethyl-1,3,2-dioxaborolan-2-yl aniline for the Suzuki reaction and the appropriate starting materials for the coupling reaction. For Examples 10-1 to 10-4, the hydrogenation step precedes the coupling step.

| | | | Retention |
|---|---|---|---|
| | | | time (min) |
| Example | Chemical Name | MS (m/z) | Method |
| 10-1 | 1-Ethyl-3[4′-hydroxy-3′-(1,1,4-trioxo-1,2,5- | (M − 1)⁻ = 403 | |
| | thiadiazolidin-2-yl)-biphenyl-3-ylmethyl]-urea | | |
| 10-2 | [4′-Hydroxy-3′-(1,1,4-trioxo-1,2,5- | (M − 1)⁻ = 390 | |
| | thiadiazolidin-2-yl)-biphenyl-3-ylmethyl]- | | |
| | carbamic acid methyl ester | | |
| 10-3 | N-[4′-Hydroxy-3′-(1,1,4-trioxo-1,2,5- | (M − 1)⁻ = 375 | |
| | thiadiazolidin-2-yl)-biphenyl-3-ylmethyl]- | | |
| | acetamide | | |
| 10-4 | [4′-Hydroxy-3′-(1,1,4-trioxo-1,2,5- | (M − 1)⁻ = 466 | |
| | thiadiazolidin-2-yl)-biphenyl-3-ylmethyl]- | | |
| | carbamic acid benzyl ester | | |
| 10-5 | 1-Ethyl-3-[4′-hydroxy-3′-(1,1,4-trioxo-1,2,5- | (M − 1)⁻ = 389 | 0.76 A |
| | thiadiazolidin-2-yl)-biphenyl-4-yl]-urea | | |

### EXAMPLE 11

### 3-[4′-Hydroxy-3′-(1,1,4-trioxo-1,2,5-thiadiazolidin-2-yl)-biphenyl-3-yl]-propionic acid

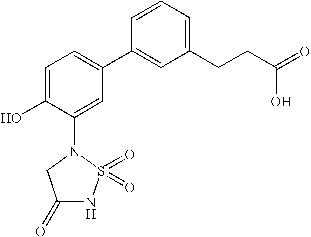

3-[4′-Hydroxy-3′-(1,1,4-trioxo-1,2,5-thiadiazolidin-2-yl)-biphenyl-3-yl]-propionic acid methyl ester is prepared according to the general procedure outlined in Example 5, using 3-(2-methoxycarbonylethyl)phenyl boronic acid. A solution of 3-[4′-hydroxy-3′-(1,1,4-trioxo-1,2,5-thiadiazolidin-2-yl)-biphenyl-3-yl]-propionic acid methyl ester (133.8 mg, 0.324 mmol) in acetonitrile (1 mL) is treated with aqueous NaOH (1 M, 0.648 mL). The solution is evaporated to dryness to afford 3-[4′-hydroxy-3′-(1,1,4-trioxo-1,2,5-thiadiazolidin-2-yl)-biphenyl-3-yl]-propionic acid: MS (M−1)⁻=375.

### EXAMPLE 12

### 5-{4-[4-Hydroxy-3-(1,1,4-trioxo-1,2,5-thiadiazolidin-2-yl)-phenyl]-pyrazol-1-yl}-pentanoic acid

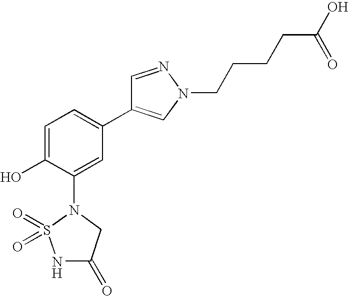

5-{4-[4-Hydroxy-3-(1,1,4-trioxo-1,2,5-thiadiazolidin-2-yl)-phenyl]-pyrazol-1-yl}-pentanoic acid is prepared analogously to Example 11: MS (M−1)⁻=393.

### EXAMPLE 13

The following compounds are prepared using appropriate starting materials and general methods described in Example 5, with the following modification: N-alkylated-pyrazolepinacolboronic ester starting materials are generated using the following procedure: 4-(4,4,5,5-tetramethyl-1,3,2-dioxaborolan-2-yl)-1H-pyrazole is added to 1 equivalent of NaH in dimethoxyethane. The appropriate alkyl bromide is added to the reaction mixture, which is then heated to 60° C. and followed by LCMS. The reaction mixture is used directly without purification in the coupling with 5-(2-benzyloxy-5-bromophenyl)-1,1-dioxo-1,2,5-thiadiazolidin-3-one (Example 1, Step E).

| Example | Chemical Name | MS (m/z) |
|---|---|---|
| 13-1 | 5-[2-Hydroxy-5-(1-propyl-1H-pyrazol-4-yl)-phenyl]-1,1- | (M − 1)⁻ = 335 |
| | dioxo-1,2,5-thiadiazolidin-3-one | |
| 13-2 | 5-[2-Hydroxy-5-(1-isobutyl-1H-pyrazol-4-yl)-phenyl]- | (M − 1)⁻ = 349 |
| | 1,1-dioxo-1,2,5-thiadiazolidin-3-one | |
| 13-3 | 5-{4-[4-Hydroxy-3-(1,1,4-trioxo-1,2,5-thiadiazolidin-2- | (M − 1)⁻ = 421 |
| | yl)-phenyl]-1H-pyrazol-1-yl}-pentanoic acid ethyl ester | |
| 13-4 | 5-{2-Hydroxy-5-[1-(4,4,4-trifluorobutyl)-1H-pyrazol-4- | (M − 1)⁻ = 403 |
| | yl]-phenyl}-1,1-dioxo-1,2,5-thiadiazolidin-3-one | |
| 13-5 | 5-{2-Hydroxy-5-[1-(3-methylbutyl)-1H-pyrazol-4-yl]- | (M − 1)⁻ = 363 |
| | phenyl}-1,1-dioxo-1,2,5-thiadiazolidin-3-one | |
| 13-6 | 5-{4-[4-Hydroxy-3-(1,1,4-trioxo-1,2,5-thiadiazolidin-2- | (M − 1)⁻ = 374 |
| | yl)-phenyl]-1H-pyrazol-1-yl}-pentanenitrile | |
| 13-7 | 4-{4-[4-Hydroxy-3-(1,1,4-trioxo-1,2,5-thiadiazolidin-2- | (M − 1)⁻ = 360 |
| | yl)-phenyl]-1H-pyrazol-1-yl}-butyronitrile | |

### EXAMPLE 14

### 5-(2-Hydroxy-5-phenoxyphenyl)-1,1-dioxo-1,2,5-thiadiazolidin-3-one

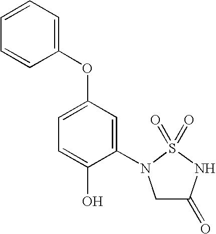

### A. 1-Benzyloxy-2-nitro-4-phenoxybenzene

To a suspension of potassium carbonate (1.14 g, 8.26 mmol) in DMF (8 mL) is added 2-nitro-4-phenoxyphenol (1.27 g, 5.5 mmol) (J Med Chem, 41, 1540) followed by benzyl bromide (0.95 g, 5.6 mmol). The mixture is stirred at RT for 18 h, then poured into water and extracted into EtOAc. The organic phase is washed with water (3×), sat. NaCl (1×), and dried over sodium sulfate. The solvent is removed under reduced pressure and the residual oil purified by flash chromatography using DCM to elute 1-benzyloxy-2-nitro-4-phenoxybenzene as a pale-yellow solid: mp=84-87° C.; ¹H-NMR (CDCl₃) δ 7.47-7.27 (m, 8H), 7.17-7.02 (m, 3H), 6.95 (d, J=8.34 Hz, 2H), 5.17 (s, 2H).

### B. 2-Benzyloxy-5-phenoxyphenylamine

To a mixture of 2-benzyloxy-2-nitro-4 phenoxybenzene (0.72 g, 2.24 mmol) and indium powder (1.0 g. 8.7 mmol) in THF (8 mL) is added conc. HCl (1.2 mL) dropwise. The mixture is stirred at RT for 2.5 h. To this mixture is added 2N NaOH which results in the formation of a gummy precipitate. The residue is triturated with EtOAc and centrifuged. The solution is decanted and the solvent removed under reduced pressure to give 2-benzyloxy-5-phenoxyphenylamine as a dark oil. This material is used directly in the next step.

### C. (2-Benzyloxy-5-phenoxyphenylamino)acetic acid methyl ester

To a mixture of 2-benzyloxy-5-phenoxyphenylamine (0.635 g, 2.18 mmol) and potassium carbonate (0.602 g, 4.36 mmol) in DMF (5 mL) is added methyl bromoacetate (0.334 g, 2.18 mmol). The mixture is stirred at 60° C. for 90 min., then an additional 150 mg of methyl bromoacetate is added and the mixture is stirred at 60° C. for 1 h. The mixture is allowed to cool to RT and then poured into water and extracted into EtOAc. The organic phase is washed with water (3×), sat. NaCl (1×), and dried over sodium sulfate. The solvent is removed under reduced pressure and the residue purified by flash chromatography using DCM to elute (2-benzyloxy-5-phenoxyphenylamino)acetic acid methyl ester as an oil: ¹H-NMR (CDCl₃) δ 7.44-7.24 (m, 7H), 7.00 (t, 1H), 6.96-6.91 (m, 2H), 6.76 (d, J=8.59 Hz, 1H), 6.28 (dd, J=8.59, 2.78 Hz, 1H), 6.21 (d, J=2.53 Hz, 1H), 5.06 (s, 2H), 4.94 (s, broad, 1H), 3.85 (s, 2H), 3.72 (s, 3H).

### D. N-(t-Butoxycarbonylsulfamoyl)-N-(2-benzyloxy-5-phenoxyphenyl)glycine methyl ester

To a solution of chlorosulfonyl isocyanate (0.23 g, 1.62 mmol) in methylene chloride (3 mL) is added dropwise a solution of t-butanol (0.12 g, 1.62 mmol) in methylene chloride (1 mL). The solution is stirred at RT for 45 min., then a solution of (2-benzyloxy-5-phenoxyphenylamino)acetic acid methyl ester (0.42 g, 1.16 mmol) and triethylamine (0.234 g, 2.34 mmol) in methylene chloride (1.5 mL) is added dropwise. The mixture is stirred at RT for 2 h, then washed with water. The organic phase is dried over sodium sulfate and the solvent removed under reduced pressure. The residual oil is purified by flash chromatography using methylene chloride to elute N-(t-butoxycarbonylsulfamoyl)-N-(2-benzyloxy-5-phenoxyphenyl)glycine methyl ester as an oil: ¹H-NMR (CDCl₃) δ 7.53-7.37 (m, 9H), 7.17 (t, 1H), 7.09-6.98 (m, 4H), 5.25 (s, 2H), 4.66 (s, 2H), 3.76 (s, 3H), 1.51 (s, 9H); MS (M−1)=541.

### E. N-Sulfamoyl-N-(2-benzyloxy-5-phenoxyphenyl)glycine methyl ester

A solution of N-(t-butoxycarbonylsulfamoyl)-N-(2-benzyloxy-5-phenoxyphenyl)glycine methyl ester (0.35 g, 0.65 mmol) in 4 mL trifluoroacetic acid/methylene chloride (1:1) is stirred at RT for 20 min. The solvent is removed under reduced pressure. Methylene chloride is added to the residue, then removed under reduced pressure. The resulting oil is purified by flash chromatography using methylene chloride to elute N-sulfamoyl-N-(2-benzyloxy-5-phenoxyphenyl)glycine methyl ester as an oil: ¹H-NMR (CDCl₃) δ 7.50-7.32 (m, 8H), 7.11 (t, 1H), 7.03-6.98 (m, 4H), 5.12 (s, 2H), 5.00 (br s, 2H), 4.37 (s, 2H), 3.68 (s, 3H); MS (M−1)=441.

### F. 5-(2-Benzyloxy-5-phenoxyphenyl)-1,1-dioxo-1,2,5-thiadiazolidin-3-one potassium salt

To a solution of N-sulfamoyl-N-(2-benzyloxy-5-phenoxyphenyl)glycine methyl ester (0.167 g, 0.38 mmol) in 2 mL of THF is added a 1.0 M solution of potassium t-butoxide (0.38 mL) in THF. The mixture is stirred at RT for 24 h and the solvent is removed under reduced pressure to give 5-(2-benzyloxy-5-phenoxyphenyl)-1,1-dioxo-1,2,5-thiadiazolidin-3-one potassium salt as a gum: MS (M−1)⁻=409. This is used directly in the next step.

### G. 5-(2-Hydroxy-5-phenoxyphenyl)-1,1-dioxo-1,2,5-thiadiazolidin-3-one

A solution of 5-(2-benzyloxy-5-phenoxyphenyl)-1,1-dioxo-1,2,5-thiadiazolidin-3-one potassium salt (0.18 g, 0.4 mmol) in water (15 mL) is hydrogenated at 1 atm over 10% Pd/C (0.05 g) for 24 h. The catalyst is filtered and the water removed by lyophilization. The residue is dissolved in a minimum volume of water and purified by preparative HPLC using a gradient of 10% acetonitrile/water to 100% acetonitrile (+0.1% TFA) over 13 min to elute 5-(2-hydroxy-5-phenoxyphenyl)-1,1-dioxo-1,2,5-thiadiazolidin-3-one as an off-white solid: mp=153-157° C.; ¹H-NMR-(DMSO-d₆) δ 9.68 (s, broad, 1H), 7.22-717 (m, 2H), 6.95-6.89 (m, 2H), 6.80-6.72 (m, 4H), 4.32 (s, 2H); MS (M−1)⁻=319.

### EXAMPLE 15

### 5-(2-Hydroxy-5-methoxyphenyl)-1,1-dioxo-1,2,5-thiadiazolidin-3-one

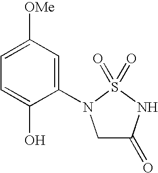

The title compound is prepared analogously to Example 14 from 4-methoxy-2-nitrophenol: ¹H-NMR (DMSO-d₆) δ 6.87 (d, J=2.78 Hz, 1H), 6.80-6.72 (m, 2H), 4.41 (s, 2H), 3.61 (s, 3H); MS (M−1)⁻=257.

### EXAMPLE 16

### 5-(5-Benzyl-2-hydroxyphenyl)-1,1-dioxo-1,2,5-thiadiazolidin-3-one

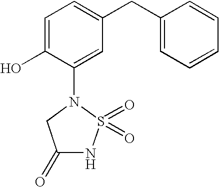

### A. 5-(5-Benzyl-2-benzyloxyphenyl)-1,1-dioxo-1,2,5-thiadiazolidin-3-one

To a microwave vial containing 5-(2-benzyloxy-5-bromophenyl)-1,1-dioxo-1,2,5-thiadiazolidin-3-one (150 mg, 0.377 mmol) and resin bound Pd tetrakis (500 mg, 0.755 mmol) in DME (5 mL) is added benzyl 9-BBN (1.51 mL, 0.755 mmol) followed by sodium carbonate (0.75 mL, 1.50 mmol). The reaction mixture is heated in the microwave for 10 min. at 110° C. The mixture is filtered through Celite to remove the resin and the filtrate is concentrated in vacuo. The crude oil is purified using reverse phase silica on the Biotage and the desired product is taken directly on to the next step.

### B. 5-(5-Benzyl-2-hydroxyphenyl)-1,1-dioxo-1,2,5-thiadiazolidin-3-one

5-(5-Benzyl-2-hydroxyphenyl)-1,1-dioxo-1,2,5-thiadiazolidin-3-one is prepared analogously to Example 1, Step G, replacing Pd/C with Pd(OH)₂: ¹H NMR (MeOD) δ 8.52 (s, 1H), 7.3 (d, J=2.27 Hz, 1H), 7.22 (m, 2H), 7.12 (m, 3H), 6.95 (dd, J=8.0, 2.0 Hz, 1H), 6.81 (d, J=8.3 Hz, 1H), 4.31 (s, 2H), 3.86 (s, 2H). Retention time=0.96 min (Method A); (M−H)⁻=317.

### EXAMPLE 17

### 5-(2-Hydroxy-5-methylphenyl)-1,1-dioxo-1,2,5-thiadiazolidin-3-one

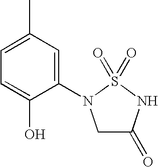

### A. 1-Benzyloxy-4-methyl-2-nitrobenzene

The title compound is prepared analogously to Example 14, Step A from 4-methyl-2-nitrophenol.

### B. 2-Benzyloxy-5-methylphenylamine

A mixture of 1-benzyloxy-4-methyl-2-nitrobenzene (2.4 g, 9.9 mmol) and PtO₂ (0.12 g) in EtOAc (45 mL) is hydrogenated at 20 psi for 1 h. The catalyst is then filtered and the filtrate is concentrated to give the title compound as an oil.

### C. 5-(2-Hydroxy-5-methylphenyl)-1,1-dioxo-1,2,5-thiadiazolidin-3-one

The title compound is prepared analogously to Example 14, Steps C-G: MS (M−1)⁻=241.

### EXAMPLE 18

### 5-(5-Hexyl-2-hydroxyphenyl)-1,1-dioxo-1,2,5-thiadiazolidin-3-one

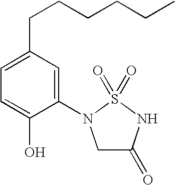

### A. 4-Benzyloxy-3-nitrobenzaldehyde

Potassium carbonate (39.75 g, 287.6 mmol) is added slowly to a solution of 4-hydroxy-3-nitrobenzaldehyde (24.03 g, 143.8 mmol) in 150 mL of DMF at ambient temperature. Benzyl bromide (25.6 mL, 36.86 g, 215.5 mmol) is added, and the mixture is warmed to 50° C. and stirred overnight. The reaction mixture is cooled to ambient temperature, water is added, and the mixture is extracted with EtOAc and diethyl ether. The organic phase is washed with water and brine, dried (Na₂SO₄) and concentrated under vacuum to afford 4-benzyloxy-3-nitrobenzaldehyde.

### B. 1-Benzyloxy-4-((Z)-hex-1-enyl)-2-nitrobenzene

To a solution of pentyltriphenylphosphonium bromide (1.34 g, 3.24 mmol) in THF (50 mL) at −20° C. is added n-BuLi (208 mg, 1.6 M in hexane) dropwise. The mixture is stirred at −20° C. for 30 min, and 4-benzyloxy-3-nitrobenzaldehyde (760 mg, 2.96 mol) in THF (5 mL) is added dropwise. After it is stirred at −20° C. for 5 min., it is allowed to warm to RT, quench with water and extracted with EtOAc. The organic layer is then washed with brine, dried with Na₂SO₄ and concentrated. The residue is purified by flash column to give the title compound as a yellow oil.

### C. 5-(5-Hexyl-2-hydroxyphenyl)-1,1-dioxo-1,2,5-thiadiazolidin-3-one

The title compound is prepared analogously to Example 17, Steps B and C: MS (M−1)⁻=311.

### EXAMPLE 19

### 5-(5-Butyl-2-hydroxyphenyl)-1,1-dioxo-1,2,5-thiadiazolidin-3-one

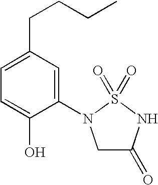

The title compound is prepared analogously to Example 18, with the exception that butyltriphenylphosphonium bromide is used in place of pentyltriphenylphosphonium bromide in Step B: MS (M−1)⁻=283.

### EXAMPLE 20

### 5-[2-Hydroxy-5-(tetrahydrofuran-3-yl)-phenyl]-1,1-dioxo-1,2,5-thiadiazolidin-3-one

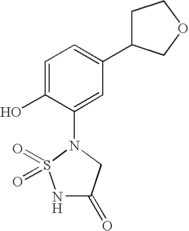

5-[2-Hydroxy-5-(tetrahydrofuran-3-yl)-phenyl]-1,1-dioxo-1,2,5-thiadiazolidin-3-one is prepared from 5-(2-benzyloxy-5-furan-3-yl-phenyl)-1,1-dioxo-1,2,5-thiadiazolidin-3-one (intermediate in the synthesis of Example 2-4) followed by hydrogenation with Pd/C: MS (M−1)⁻=297.

### EXAMPLE 21

### 5-[5-(4-Fluorophenylethynyl)-2-hydroxyphenyl]-1,1-dioxo-1,2,5-thiadiazolidin-3-one

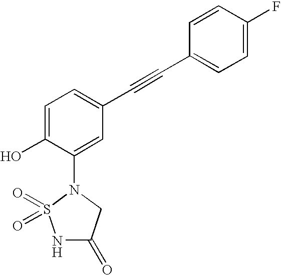

### A. 5-[2-Benzyloxy-5-(4-fluorophenylethynyl)-phenyl]-1,1-dioxo-1,2,5-thiadiazolidin-3-one

A solution of 5-(2-benzyloxy-5-bromophenyl)-1,1-dioxo-1,2,5-thiadiazolidin-3-one (Example 1, Step E) (96.2 mg, 0.242 mmol) in dimethoxyethane (4 mL) is stirred with aqueous sodium carbonate (2 M, 0.484 mL, 0.968 mmol), dichloro[1,1′-bis(diphenylphosphino)ferrocene]-palladium (II) dichloromethane adduct (19.8 mg, 0.024 mmol), copper (I) iodide (9.2 mg, 0.048 mmol, 20 mol %), and 1-ethynyl-4-fluorobenzene (79.0 μL, 0.484 mmol) at 80° C. for 16 h. 1N HCl is added and the suspension is extracted with EtOAc. The organic layer is washed with saturated sodium chloride, dried over Na₂SO₄, filtered and evaporated to dryness to afford 5-[2-benzyloxy-5-(4-fluorophenylethynyl)-phenyl]-1,1-dioxo-1,2,5-thiadiazolidin-3-one: MS (M−1)⁻=435.

### B. 5-[5-(4-Fluorophenylethynyl)-2-hydroxyphenyl]-1,1-dioxo-1,2,5-thiadiazolidin-3-one

To a −78° C. solution of 5-[2-benzyloxy-5-(4-fluorophenylethynyl)-phenyl]-1,1-dioxo-1,2,5-thiadiazolidin-3-one (17.4 mg, 0.040 mmol) in DCM (1 mL) is added boron tribromide (1 M in DCM, 47.9 μL, 0.0479 mmol). The reaction is warmed to 23° C. over 20 min. and quenched with 1N HCl (1 mL). The resulting suspension is extracted with EtOAc. The organic layer is dried over Na₂SO₄, filtered and evaporated to dryness to afford 5-[5-(4-fluorophenylethynyl)-2-hydroxyphenyl]-1,1-dioxo-1,2,5-thiadiazolidin-3-one: MS (M−1)⁻=345.

### EXAMPLE 22

The following compounds are prepared using appropriate starting materials and general methods described in Example 21.

| Example | Chemical Name | MS (m/z) |
|---|---|---|
| 22-1 | 6-[4-Hydroxy-3-(1,1,4-trioxo-1,2,5-thiadiazolidin-2-yl)- | (M − 1)⁻ = 318 |
| | phenyl]-hex-5-ynenitrile | |
| 22-2 | 6-[4-Hydroxy-3-(1,1,4-trioxo-1,2,5-thiadiazolidin-2-yl)- | (M − 1)⁻ = 337 |
| | phenyl]-hex-5-ynoic acid | |
| 22-3 | 5-[5-(3,3-Dimethyl-but-1-ynyl)-2-hydroxyphenyl]-1,1- | (M − 1)⁻ = 307 |
| | dioxo-1,2,5-thiadiazolidin-3-one | |

### EXAMPLE 23

### 5-[2-Hydroxy-5-(5-methylhexyl)-phenyl]-1,1-dioxo-1,2,5-thiadiazolidin-3-one

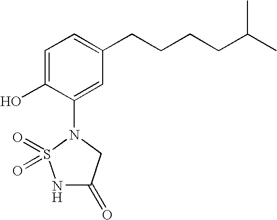

### A. 5-[2-Benzyloxy-5-(5-methylhex-1-ynyl)-phenyl]-1,1-dioxo-1,2,5-thiadiazolidin-3-one

5-[2-Benzyloxy-5-(5-methylhex-1-ynyl)-phenyl]-1,1-dioxo-1,2,5-thiadiazolidin-3-one is prepared analogously to Example 21, Step A using 5-methylhex-1-yne with the exception that the reaction is performed in the microwave at 110° C. for 20 min.

### B. 5-[2-Hydroxy-5-(5-methylhexyl)-phenyl]-1,1-dioxo-1,2,5-thiadiazolidin-3-one

5-[2-Hydroxy-5-(5-methylhexyl)-phenyl]-1,1-dioxo-1,2,5-thiadiazolidin-3-one is prepared analogously to Example 1, Step G, with the exception that Pd(OH)₂ is used in place of Pd/C: MS (M−1)⁻=325.

### EXAMPLE 24

### 6-[4-Hydroxy-3-(1,1,4-trioxo-1,2,5-thiadiazolidin-2-yl)-phenyl]-hexanoic acid

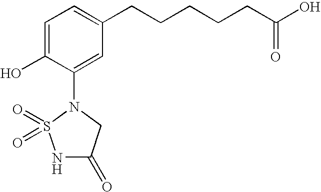

A suspension of 6-[4-benzyloxy-3-(1,1,4-trioxo-1,2,5-thiadiazolidin-2-yl)-phenyl]-hex-5-ynoic acid (Example 22-2) and Pd/C (10 wt %, 39 mg) in water (30 mL) is stirred under an atmosphere of H₂ for 3 h. The suspension is filtered and evaporated to dryness to afford 6-[4-hydroxy-3-(1,1,4-trioxo-1,2,5-thiadiazolidin-2-yl)-phenyl]-hexanoic acid: MS (M−1)⁻=341.

### EXAMPLE 25

### 5-[5-(Benzylaminomethyl)-2-hydroxyphenyl]-1,1-dioxo-1,2,5-thiadiazolidin-3-one

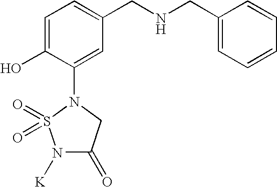

### A. 4-Benzyloxy-3-nitrobenzaldehyde

The title compound is prepared as described in Example 18, Step A.

### B. Benzyl-(4-benzyloxy-3-nitrobenzyl)-amine

Benzylamine (2.2 mL, 2.16 g, 20.16 mmol) is added to a solution of 4-benzyloxy-3-nitrobenzaldehyde (4.31 g, 16.77 mmol) in 50 mL of 1,2-dichloroethane (DCE) at ambient temperature. Two hours later, sodium triacetoxyborohydride (10.66 g, 50.31 mmol) is added, followed by an additional 20 mL of DCE. The reaction is quenched by the addition of 1N aqueous HCl sufficient to adjust the pH to 5. The mixture is then stirred for 20 min., basified to pH 11, and extracted with EtOAc. The organic solution is dried (Na₂SO₄) and concentrated under vacuum to afford benzyl-(4-benzyloxy-3-nitrobenzyl)amine as a yellow solid.

### C. (3-Amino-4-benzyloxybenzyl)-benzylcarbamic acid benzyl ester

Benzyl chloroformate (2.485 mL, 3.01 g, 17.65 mmol) is added to a solution of benzyl-(4-benzyloxy-3-nitrobenzyl)amine and 1N NaOH (50 mL) in dioxane (50 mL) at RT. The mixture is partitioned between water and ether, and the ether solution is dried (Na₂SO₄) and concentrated to give the crude product. The product is purified by chromatography on silica gel (40% EtOAc in hexane as eluent) to afford the product as a yellow oil.

This product is stirred in EtOAc (50 mL) with platinum oxide (0.8 g) under hydrogen (1 atm) for 6 h. The mixture is filtered, concentrated and chromatographed on silica gel (30% EtOAc in hexane as eluent) to afford (3-amino-4-benzyloxybenzyl)-benzylcarbamic acid benzyl ester as a pale yellow oil.

### D. {5-[(Benzyl-benzyloxycarbonylamino)-methyl]-2-benzyloxyphenylamino}-acetic acid methyl ester

A mixture of (3-amino-4-benzyloxybenzyl)-benzylcarbamic acid benzyl ester (0.503 g, 1.11 mmol), methyl bromoacetate (0.17 g, 1.11 mmol), and potassium carbonate (0.233 g, 1.68 mmol) in DMF (3 mL) is stirred at ambient temperature overnight. The mixture is taken up in EtOAc and washed with water and brine, dried (Na₂SO₄) and concentrated to afford crude product. Chromatography on silica gel (30% EtOAc in hexane as eluent) affords 365 mg of {5-[(benzylbenzyloxycarbonylamino)-methyl]-2-benzyloxyphenylamino}-acetic acid methyl ester as a pale yellow solid.

### E. N-(t-Butoxycarbonylsulfamoyl)-N-((5-benzylbenzyloxycarbonylaminomethyl)-2-benzyloxyphenyl)-acetic acid methyl ester

Chlorosulfonyl isocyanate (0.129 g, 0.91 mmol) is added to a solution of t-butyl alcohol (0.067 g, 0.905 mmol) in DCM (3 mL) at ambient temperature. The solution is stirred for 2 h, after which a mixture of {5-[(benzylbenzyloxycarbonylamino)-methyl]-2-benzyloxyphenylamino}-acetic acid methyl ester (365 mg, 0.696 mmol) and Et₃N (0.12 g, 1.184 mmol) in 3 mL of DCM is added. This mixture is stirred overnight and then washed with water and brine. The organic solution is dried (Na₂SO₄) and concentrated to give crude product. The crude product is chromatographed on silica gel (30% EtOAc in hexane as eluent) to afford 0.2 g of N-(t-butoxycarbonylsulfamoyl)-N-((5-benzylbenzyloxycarbonylaminomethyl)-2-benzyloxyphenyl)-acetic acid methyl ester as a colorless oil.

### F. N-Sulfamoyl-N-((5-benzylbenzyloxycarbonylaminomethyl)-2-benzyloxy-phenyl)-acetic acid methyl ester

N-(t-Butoxycarbonylsulfamoyl)-N-((5-benzylbenzyloxycarbonylaminomethyl)-2-benzyloxyphenyl)-acetic acid methyl ester (0.2 g, 0.286 mmol) is stirred in 4 mL of a 1:1 mixture of DCM and trifluoroacetic acid at ambient temperature for 2 h. The mixture is concentrated under vacuum, taken up in DCM and concentrated again. This process is repeated two more times. The crude product is chromatographed on silica gel using 30% EtOAc in hexane as eluent to afford 112 mg of N-sulfamoyl-N-((5-benzylbenzyloxycarbonylaminomethyl)-2-benzyloxyphenyl)-acetic acid methyl ester.

### G. Benzyl-[4-benzyloxy-3-(1,1,4-trioxo-1,2,5-thiadiazolidin-2-yl)-benzyl]-carbamic acid benzyl ester

Potassium t-butoxide (1 M in THF, 0.37 mL) is added to a solution of N-sulfamoyl-N-((5-benzylbenzyloxycarbonylaminomethyl)-2-benzyloxyphenyl)-acetic acid methyl ester (111 mg, 0.184 mmol) in 1 mL of THF at ambient temperature. The reaction is stirred 2 h, then quenched with 1 mL of 1N aqueous HCl. The mixture is evaporated to dryness under vacuum and purified by flash chromatography using 20% EtOH in DCM to give 100 mg of product. The potassium salt is regenerated by adding potassium t-butoxide (1 M in THF, 0.129 mL) to the product, followed by evaporation to dryness under vacuum to afford the salt of benzyl-[4-benzyloxy-3-(1,1,4-trioxo-1,2,5-thiadiazolidin-2-yl)-benzyl]-carbamic acid benzyl ester.

### H. 5-[5-(Benzylaminomethyl)-2-hydroxyphenyl]-1,1-dioxo-1,2,5-thiadiazolidin-3-one

5-[5-(Benzylaminomethyl)-2-hydroxyphenyl]-1,1-dioxo-1,2,5-thiadiazolidin-3-one is prepared analogously to Example 1, Step G: MS (M+1)⁺=348.

### EXAMPLE 26

The following compounds are prepared using appropriate starting materials and general methods described in Example 25.

| Example | Chemical Name | MS (m/z) |
|---|---|---|
| 26-1 | 5-(5-Butylaminomethyl-2-hydroxyphenyl)-1,1-dioxo-1,2,5- | (M + 1)⁺ = 314 |
| | thiadiazolidin-3-one | |
| 26-2 | 5-{2-Hydroxy-5-[(2-methoxybenzylamino)-methyl]-phenyl}- | (M + 1)⁺ = 378 |
| | 1,1-dioxo-1,2,5-thiadiazolidin-3-one | |
| 26-3 | 5-{5-[(2-Ethoxybenzylamino)-methyl]-2-hydroxyphenyl}-1,1- | (M + 1)⁺ = 392 |
| | dioxo-1,2,5-thiadiazolidin-3-one | |
| 26-4 | 5-{2-Hydroxy-5-[(2-isopropoxybenzylamino)-methyl]-phenyl}- | (M + 1)⁺ = 406 |
| | 1,1-dioxo-1,2,5-thiadiazolidin-3-one | |
| 26-5 | 5-(2-Hydroxy-5-{[2-(1-methyl-2-phenylethoxy)-benzylamino]- | (M + 1)⁺ = 482 |
| | methyl}-phenyl)-1,1-dioxo-1,2,5-thiadiazolidin-3-one | |

### EXAMPLE 27

### 5-[2-Hydroxy-5-(3-methylbutoxy)-phenyl]-1,1-dioxo-1,2,5-thiadiazolidin-3-one

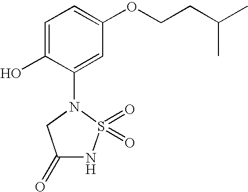

### A. Acetic acid 4-benzyloxyphenyl ester

To a stirred solution of 4-benzyloxyphenol (20 g, 0.10 mol) in pyridine (200 mL) is added acetic anhydride (20.4 g, 0.20 mol) followed by catalytic amount of DMAP. The mixture is stirred at RT for 2 h. The reaction product is diluted with EtOAc and washed with 1N HCl, saturated NaCl, and finally with water. The organic phase is dried over anhydrous MgSO₄, filtered and concentrated to afford acetic acid 4-benzyloxyphenyl ester.

### B. Acetic acid 4-benzyloxy-3-nitrophenyl ester

Acetic acid 4-benzyloxyphenyl ester (15 g, 61.9 mmol) is dissolved in DCM (350 mL). Conc. HNO₃ over silica gel is added and the suspension is stirred at RT 4 h. The silica gel is filtered and washed with DCM. The solvent is evaporated and EtOAc is added to the filtrate, followed by saturated sodium bicarbonate. The organic phase is washed with water (3×) and EtOAc, and concentrated to yield a yellow solid. The solid is washed with ether, filtered and washed again with ether to afford acetic acid 4-benzyloxy-3-nitrophenyl ester.

### C. 4-Benzyloxy-3-nitrophenol

Potassium carbonate (3 g) is added to a solution of acetic acid 4-benzyloxy-3-nitrophenyl ester (4.23 g, 14.7 mmol) in MeOH/THF (60 mL/30 mL) and the mixture is stirred at RT for 1.5 h. Ethyl acetate is added to the mixture and the mixture is washed with 1N HCl followed by saturated sodium chloride. The organic phase is dried over MgSO₄, filtered and concentrated to afford 4-benzyloxy-3-nitrophenol.

### D. 1-Benzyloxy-4-(3-methyl-but-2-enyloxy)-2-nitrobenzene

1-Benzyloxy-4-(3-methylbut-2-enyloxy)-2-nitrobenzene is prepared analogously to Example 25, Step D, starting with 1-bromo-3-methyl-but-2-ene.

### E. 2-Benzyloxy-5-(3-methylbutoxy)-phenylamine

2-Benzyloxy-5-(3-methylbutoxy)-phenylamine is prepared analogously to Example 17, Step B, using Pt/C in place of PtO₂.

### F. 2-Benzyloxy-5-(3-methylbutoxyphenyl)-N-(t-butoxycarbonylsulfamoyl)glycine tert-butyl ester

2-Benzyloxy-5-(3-methylbutoxyphenyl)-N-(t-butoxycarbonylsulfamoyl)glycine tert-butyl ester is prepared following the general procedures outlined in Example 25, Steps D and E.

### G. 2-Benzyloxy-5-(3-methylbutoxy)-phenyl)-N-(t-butoxycarbonylsulfamoyl-N′-trimethylsilanylethyl)-acetic acid tert-butyl ester

A solution of 2-benzyloxy-5-(3-methylbutoxyphenyl)-N-(t-butoxycarbonylsulfamoyl)glycine tert-butyl ester (1.4 g, 2.42 mmol) and 2-trimethylsilanylethanol (0.56 g, 4.7 mmol) in toluene (40 mL) is cooled down to 0° C. Triphenyl phosphine (1.5 g, 5.72 mmol) and diisopropyl azodicarboxylate (1.14 g, 5.63 mmol) are added. The ice bath is removed and the mixture is stirred at RT overnight. The mixture is concentrated to remove toluene. The crude material is dissolved in hexane and the triphenyl phosphine oxide by-product is filtered off. The filtrate is concentrated and purified by flash chromatography to afford 2-benzyloxy-5-(3-methylbutoxy)-phenyl)-N-(t-butoxycarbonylsulfamoyl-N′-trimethylsilanylethyl)-acetic acid tert-butyl ester.

### H. [[2-Benzyloxy-5-(3-methylbutoxy)-phenyl]-N-sulfamoyl-(N′-trimethylsilanylethy)] carbamic acid

[[2-Benzyloxy-5-(3-methylbutoxy)-phenyl]-N-sulfamoyl-(N′-trimethylsilanyl-ethyl) ] carbamic acid is prepared analogously to Example 25, Step F.

### I. 5-[2-Benzyloxy-5-(3-methylbutoxy)-phenyl]-1,1-dioxo-2-(2-trimethylsilanylethyl)-1,2,5-thiadiazolidin-3-one

1-Hydroxy-7-azabenzotriazole (HOAt) and 1-(3-dimethylaminopropyl)-3-ethylcarbodiimide hydrochloride (EDCI) are added to a solution of [[2-benzyloxy-5-(3-methyl-butoxy)-phenyl]-N-sulfamoyl-(N′-trimethylsilanylethyl)] carbamic acid (1.1 g, 1.75 mmol) in THF (25 mL). The reaction mixture is stirred at RT for 5 min. and triethylamine is added to the suspension. The mixture is stirred at RT overnight. Ethyl acetate is added to the suspension and washed with 1N HCl and then water. The organic phase is dried over MgSO₄, filtered and concentrated. The crude material is purified by flash chromatography to afford 5-[2-benzyloxy-5-(3-methylbutoxy)-phenyl]-1,1-dioxo-2-(2-trimethylsilanylethyl)-1,2,5-thiadiazolidin-3-one.

### J. 5-[2-Benzyloxy-5-(3-methylbutoxy)-phenyl]-1,1-dioxo-1,2,5-thiadiazolidin-3-one

To a solution of 5-[2-benzyloxy-5-(3-methylbutoxy)-phenyl]-1,1-dioxo-2-(2-trimethylsilanylethyl)-1,2,5-thiadiazolidin-3-one (460 mg, 0.91 mmol) in THF (16 mL) is added TBAF (0.5 M in THF, 3.48 mL, 1.81 mmol) and the mixture is refluxed for 1.5 h. The reaction mixture is washed with 1N HCl solution (4×) and brine (1×) after the addition of EtOAc. It is then dried with MgSO₄ and concentrated to give the title compound.

### K. 5-[2-Hydroxy-5-(3-methylbutoxy)-phenyl]-1,1-dioxo-1,2,5-thiadiazolidin-3-one

5-[2-Hydroxy-5-(3-methylbutoxy)-phenyl]-1,1-dioxo-1,2,5-thiadiazolidin-3-one is prepared following the general procedure outlined in Example 1, Step G: MS (M−1)⁻=313.

### EXAMPLE 28

### 5-[2-Hydroxy-5-(4-methylpentyloxy)-phenyl]-1,1-dioxo-1,2,5-thiadiazolidin-3-one

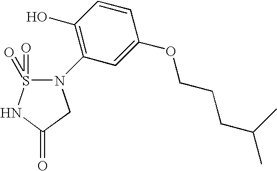

5-[2-Hydroxy-5-(4-methylpentyloxy)-phenyl]-1,1-dioxo-1,2,5-thiadiazolidin-3-one is prepared analogously to Example 27, using 1-bromo-4-methylpentane in Step D: MS (M−1)=327.

### EXAMPLE 29

### 5-(2-Hydroxy-5-propoxyphenyl)-1,1-dioxo-1,2,5-thiadiazolidin-3-one

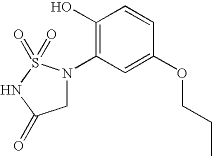

5-(2-Hydroxy-5-propoxyphenyl)-1,1-dioxo-1,2,5-thiadiazolidin-3-one is prepared analogously to Example 27, with the following changes: the starting material used for Step D is 3-bromopropene, and the nitro reduction of Step E is carried out using iron in AcOH/EtOH (Example 1, Step B) to afford the aniline: MS (M−1)=285.

### EXAMPLE 30

### 2-Hydroxy-6-{4-[4-hydroxy-3-(1,1,4-trioxo-1,2,5-thiadiazolidin-2-yl)-phenyl]-butoxy}-N,N-dimethylbenzamide

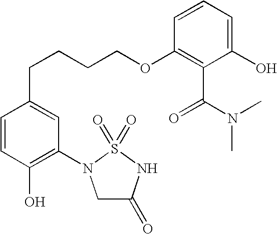

### A. 2-But-3-enyloxy-6-hydroxybenzoic acid methyl ester

Prepared from methyl 2,6-dihydroxybenzoate and but-3-en-1-ol analogously to Example 27, Step G, using DEAD in place of DIAD: MS (M−1)⁻=221.

### B. 2-Benzyloxy-6-but-3-enyloxybenzoic acid methyl ester

Prepared from 2-but-3-enyloxy-6-hydroxybenzoic acid methyl ester analogously to Example 1, Step A.

### C. 2-Benzyloxy-6-but-3-enyloxybenzoic acid

A mixture of 2-benzyloxy-6-but-3-enyloxybenzoic acid methyl ester (1.2 g, 3.68 mmol) and NaOH (589 mg, 6N solution) in water (2.5 mL), MeOH (6 mL) and THF (20 mL) is heated at 60° C. for 24 h, then at 90° C. for 5 days. After the solvent is removed, the residue is acidified with 1N HCl solution to pH 2. EtOAc is added to extract and the organic phase is washed with water and brine. It is then dried and concentrated to give the title compound as a yellow liquid.

### D. 2-Benzyloxy-6-but-3-enyloxybenzoylchloride

To a stirred solution of 2-benzyloxy-6-but-3-enyloxybenzoic acid (800 mg, 2.45 mmol) in 15 mL DCM and 1 drop DMF is added oxalyl chloride (0.86 mL, 9.82 mmol). The solution is stirred at ambient temperature overnight. The solvent is removed under pressure and the residue is dissolved in DCM, then the DCM is removed under reduced pressure again and repeated 3× to give the product as a yellow liquid.

### E. 2-Benzyloxy-6-but-3-enyloxy-N,N-dimethylbenzamide

A mixture of 2-benzyloxy-6-but-3-enyloxybenzoylchloride (1.2 g, 3.6 mmol) and dimethylamine (2N in THF, 10.9 mL, 21.8 mmol) in THF (20 mL) is stirred at ambient temperature for 18 h. After the solvent is removed, water is added and EtOAc is used to extract. The organic phase is then washed with water, brine and dried. It is then concentrated to give the title compound as a red liquid.

### F. 2-Benzyloxy-6-{4-[4-benzyloxy-3-(1,1,4-trioxo-1,2,5-thiadiazolidin-2-yl)-phenyl]-butoxy}-N,N-dimethylbenzamide

To a stirred solution of 2-benzyloxy-6-but-3-enyloxy-N,N-dimethylbenzamide (112 mg, 0.35 mmol) in 2 mL of THF at 0° C. is added 9-BBN (0.72 mL, 0.36 mmol, 0.5 M in THF). The solution is warmed to ambient temperature and stirred overnight. The solvent is removed under reduced pressure and the residue is dissolved in 4 mL of DME and 0.5 mL of H₂O. To this solution is added 5-(2-benzyloxy-5-bromophenyl)-1,1-dioxo-1-1,2,5-thiadiazolidin-3-one (Example 1, Step E; 114 mg, 0.29 mmol), Pd(PPh₃)₄ (10 mg) and K₂CO₃ (120 mg, 0.87 mmol). The mixture is microwaved at 120° C. for 50 minutes. The suspension is filtered and solvent is removed under reduced pressure to give the product as a dark red liquid: MS (M−1)⁻=642.

### G. 2-Hydroxy-6-{4-[4-hydroxy-3-(1,1,4-trioxo-1,2,5-thiadiazolidin-2-yl)-phenyl]-butoxy}-N,N-dimethylbenzamide

Prepared from 2-benzyloxy-6-{4-[4-benzyloxy-3-(1,1,4-trioxo-1,2,5-thiadiazolidin-2-yl)-phenyl]-butoxy}-N,N-dimethylbenzamide analogously to Example 1, Step G: MS (M−1)⁻=462.

### EXAMPLE 31

The following compounds are prepared using appropriate starting materials and general methods described in Example 30.

| Example | Chemical Name | MS (m/z) |
|---|---|---|
| 31-1 | 2-Hydroxy-6-{5-[4-hydroxy-3-(1,1,4-trioxo-1,2,5- | (M − 1)⁻ = 476 |
| | thiadiazolidin-2-yl)-phenyl]-pentyloxy}-N,N- | |
| | dimethylbenzamide | |
| 31-2 | 2-Hydroxy-6-{6-[4-hydroxy-3-(1,1,4-trioxo-1,2,5- | (M − 1)⁻ = 490 |
| | thiadiazolidin-2-yl)-phenyl]-hexyloxy}-N,N- | |
| | dimethylbenzamide | |
| 31-3 | 2-Fluoro-6-{6-[4-hydroxy-3-(1,1,4-trioxo-1,2,5-thiadiazolidin- | (M − 1)⁻ = 492 |
| | 2-yl)-phenyl]-hexyloxy}-N,N-dimethylbenzamide | |
| 31-4 | 2-Hydroxy-6-{7-[4-hydroxy-3-(1,1,4-trioxo-1,2,5- | (M − 1)⁻ = 504 |
| | thiadiazolidin-2-yl)-phenyl]-heptyloxy}-N,N- | |
| | dimethylbenzamide | |

### EXAMPLE 32

### 5-(4-Hydroxy-4′-hydroxymethylbiphenyl-3-yl)-1,1-dioxo-1,2,5-thiadiazolidin-3-one

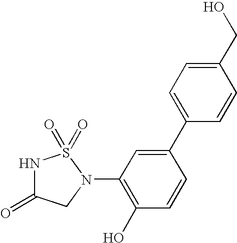

### A. 5-[2-Benzyloxy-5-(4,4,5,5-tetramethyl-[1,3,2]dioxaborolan-2-yl)-phenyl]-1,1-dioxo-1,2,5-thiadiazolidin-3-one

To a stirred suspension of 5-(2-benzyloxy-5-bromophenyl)-1,1-dioxo-1-1,2,5-thiadiazolidin-3-one (Example 1, Step E) (100 mg, 0.25 mmol), bis(pinacolato)diboron (127 mg, 0.50 mmol) and CH₃COOK (74 mg, 0.75 mmol) in 3 mL of DMF is added Pd(dppf)Cl₂ (10 mg, 10% weight). The suspension is degassed and heated at 100° C. overnight. The mixture is filtered and the filtrate is used directly for the next step: MS (M−1)⁻=443.

### B. 5-(4-Benzyloxy-4′-hydroxymethylbiphenyl-3-yl)-1,1-dioxo-1,2,5-thiadiazolidin-3-one

To the above solution is added (4-iodophenyl)-methanol (88 mg, 0.38 mmol), Cs₂CO₃ (326 mg, 1.0 mmol) and Pd(PPh₃)₄ (10 mg, 10% weight). The suspension is degassed and heated at 85° C. for 3 h. The solvent is removed under reduced pressure to give the product as a red liquid: MS (M−1)⁻=423.

### C. 5-(4-Hydroxy-4′-hydroxymethylbiphenyl-3-yl)-1,1-dioxo-1,2,5-thiadiazolidin-3-one

Prepared from 5-(4-benzyloxy-4′-hydroxymethylbiphenyl-3-yl)-1,1-dioxo-1,2,5-thiadiazolidin-3-one analogously to Example 1, Step G: MS (M−1)⁻=333.

### EXAMPLE 33

### 5-(2-Hydroxy-4,5-dimethylphenyl)-1,1-dioxo-1,2,5-thiadiazolidin-3-one

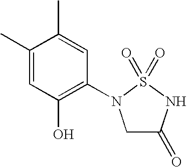

### A. N-(2-Hydroxy-4,5-dimethylphenyl)acetamide

To a stirred suspension of 2-amino-4,5-dimethylphenol (450 mg, 3.28 mmol) in 10 mL of THF and 10 mL saturated NaHCO₃ is added acetyl chloride (0.25 mL, 3.45 mmol) dropwise at 0° C. The suspension is warmed to ambient temperature and stirred for 2 hours. The suspension is filtered and the filtrate is adjusted to pH 4-5 with 1N HCl. The suspension is extracted with EtOAc. The organic layer is washed with water and brine, and is dried over sodium sulfate. The solvent is removed under reduced pressure to give the crude product as a pale yellow solid: NMR (CDCl₃): δ 8.47 (s, 1H), 7.32 (s, 1H), 6.80 (s, 1H), 6.68 (s, 1H), 2.23 (s, 3H), 2.18 (s, 3H), 2.14 (s, 3H); MS (M−1)⁻=178.

### B. N-(2-Benzyloxy-4,5-dimethylphenyl)-acetamide

Prepared from N-(2-hydroxy-4,5-dimethylphenyl)acetamide analogously to Example 1, Step A: MS (M−1)⁻=268.

### C. 2-Benzyloxy-4,5-dimethylphenylamine

N-(2-Benzyloxy-4,5-dimethylphenyl)acetamide (800 mg, 2.97 mmol) is refluxed in 6 mL EtOH with KOH (999 mg, 17.8 mmol) in water (2 mL) overnight. The solution is diluted with water and extracted with EtOAc. The organic layer is washed with water and brine, and is dried over sodium sulfate. The solvent is removed under reduced pressure to give the product as a pale red liquid: NMR (CDCl₃): δ 7.44-7.30 (m, 5H), 6.67 (s, 1H), 6.55 (s, 1H), 5.04 (s, 2H), 3.64 (s, 2H), 2.15 (s, 3H), 2.13 (s, 3H); MS (M+1)⁺=228.

### D. 5-(2-Hydroxy-4,5-dimethylphenyl)-1,1-dioxo-1,2,5-thiadiazolidin-3-one

Prepared from 2-benzyloxy-4,5-dimethylphenylamine analogously to Example 14, Steps C-G: MS (M−1)⁻=255.

### EXAMPLE 34

### 5-[4-Hydroxy-3-(1,1,4-trioxo-1,2,5-thiadiazolidin-2-yl)-phenyl]-2,2-dimethylpentanoic acid

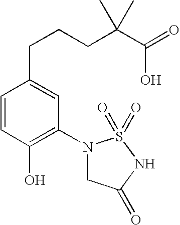

The title compound is prepared analogously to Example 30, Step F and G starting with 2,2-dimethylpent-4-enoic acid in Step F: MS (M−1)⁻=355.

### EXAMPLE 35

### 8-[4-Hydroxy-3-(1,1,4-trioxo-1,2,5-thiadiazolidin-2-yl)-phenyl]-2,2-dimethyloctanoic acid ethyl ester

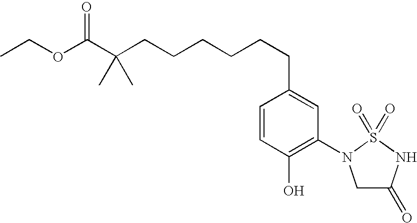

### A. 2,2-Dimethyloct-7-enoic acid ethyl ester

To a solution of isobutyric acid ethyl ester (1.0 g, 8.62 mmol) in THF (2 mL) at −78° C. is added LDA (2 M in THF, 4.31 mL, 8.62 mmol) in THF (5 mL) and the resulting mixture is allowed to warm to RT and stirred for 20 min. It is then re-cooled to −78° C. and 6-bromohex-1-ene (1.4 g, 8.62 mmol) in THF (2 mL) is added. The mixture is then allowed to warm to RT and stirred at 40° C. for 24 h. Water is added and EtOAc is used to extracted. The organic layer is washed with water, brine and dried. The concentrated residue is then purified by flash chromatography to give the title compound as a yellow liquid.

### B. 8-[4-Hydroxy-3-(1,1,4-trioxo-1,2,5-thiadiazolidin-2-yl)-phenyl]-2,2-dimethyloctanoic acid ethyl ester

The title compound is prepared analogously to Example 30, Steps F and G, with the exception that Pd(OAc)₂, 2-(di-t-butylphosphine)biphenyl and triethylamine is used in place of Pd(PPh₃)₄ and K₂CO₃; MS (M−1)⁻=425.

### EXAMPLE 36

### 8-[4-Hydroxy-3-(1,1,4-trioxo-1,2,5-thiadiazolidin-2-yl)-phenyl]-2,2-dimethyloctanoic acid

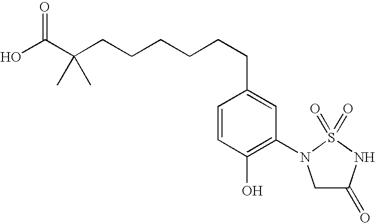

### A. 2,2-Dimethyloct-7-enoic acid

The title compound is prepared analogously to Example 30, Step C.

### B. 8-[4-Hydroxy-3-(1,1,4-trioxo-1,2,5-thiadiazolidin-2-yl)-phenyl]-2,2-dimethyloctanoic acid

The title compound is prepared analogously to Example 35, Step B: MS (M−1)⁻=397.

### EXAMPLE 37

### 7-[4-Hydroxy-3-(1,1,4-trioxo-1,2,5-thiadiazolidin-2-yl)-phenyl]-2,2-dimethylheptanoic acid

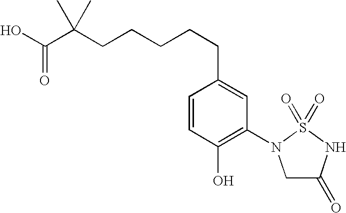

The title compound is prepared analogously to Example 36, starting from 5-bromopent-1-ene: MS (M−1)⁻=383.

### EXAMPLE 38

### 6-[4-Hydroxy-3-(1,1,4-trioxo-1,2,5-thiadiazolidin-2-yl)-phenyl]-2,2-dimethylhexanoic acid

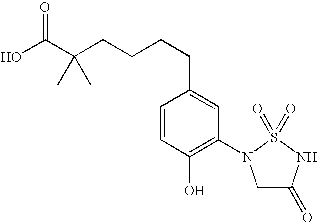

The title compound is prepared analogously to Example 36 starting from isobutyric acid ethyl ester, MS (M−1)⁻=369.

### EXAMPLE 39

### 7-[4-Hydroxy-3-(1,1,4-trioxo-1,2,5-thiadiazolidin-2-yl)-phenyl]-2,2-dimethylheptanoic acid ethyl ester

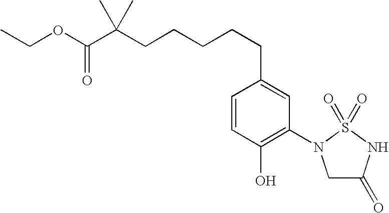

The title compound is prepared analogously to Example 35, starting from 5-bromopent-1-ene: MS (M−1)⁻=411.

### EXAMPLE 40

### 8-[4-Hydroxy-3-(1,1,4-trioxo-1,2,5-thiadiazolidin-2-yl)-phenyl]-2,2-dimethyloctanenitrile

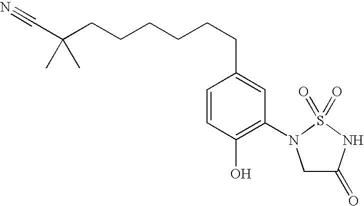

The title compound is prepared analogously to Example 35, starting from isobutyronitrile: MS (M−1)⁻=411.

### EXAMPLE 41

### 5-[2-Hydroxy-5-(6-hydroxy-6-methylheptyl)-phenyl]-1,1-dioxo-1,2,5-thiadiazolidin-3-one

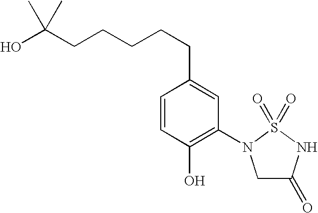

### A. 2-Methylhept-6-en-2-ol

To magnesium (89 mg, 3.70 mmol) in THF (10 mL) is added 5-bromopent-1-ene (500 mg, 3.36 mmol) dropwise and the mixture is refluxed for 2 h. After it is cooled to −78° C., acetone (0.25 mL, 3.36 mol) is added dropwise. The mixture is then stirred at ambient temperature for 18 h. 5% HCl solution is added and the EtOAc is used to extract. The organic layer is washed with water, brine and dried. It is then concentrated to give the title compound as a pale yellow liquid.

### B. 5-[2-Hydroxy-5-(6-hydroxy-6-methylheptyl)-phenyl]-1,1-dioxo-1,2,5-thiadiazolidin-3-one

The title compound is prepared analogously to Example 35, Step B: MS (M−1)⁻=355.

### EXAMPLE 42

### 5-[2-Hydroxy-5-(7-hydroxy-6,6-dimethylheptyl)-phenyl]-1,1-dioxo-1,2,5-thiadiazolidin-3-one

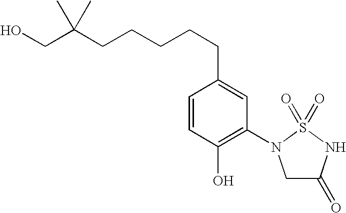

### A. 2,2-Dimethylhept-6-en-1-ol

To LiAlH₄ (1 M in THF, 6.74 mL, 6.74 mmol) in THF (10 mL) at 0° C. is added 2,2-dimethylheptanoic acid ethyl ester (intermediate from Example 37) (700 mg, 4.49 mmol) in THF (5 mL) and the mixture is stirred at ambient temperature for 18 h. Water is added and the mixture is extracted with EtOAc. The organic layer is washed with water and brine. It is then dried and concentrated to give the title compound as a yellow liquid.

### B. 7-[4-Hydroxy-3-(1,1,4-trioxo-1,2,5-thiadiazolidin-2-yl)-phenyl]-2,2-dimethylheptanoic acid

The title compound is prepared analogously to Example 35, Step B: MS (M−1)⁻=369.

### EXAMPLE 43

### 5-[2-Hydroxy-5-(5-hydroxy-5-methylhexyl)-phenyl]-1,1-dioxo-1,2,5-thiadiazolidin-3-one

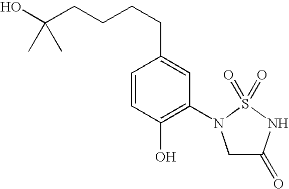

The title compound is prepared analogously to Example 41 starting with 4-bromobut-1-ene: MS (M−1)⁻=341.

### EXAMPLE 44

### 5-[2-hydroxy-5-(8-hydroxy-7,7-dimethyloctyl)-phenyl]-1,1-dioxo-1,2,5-thiadiazolidin-3-one

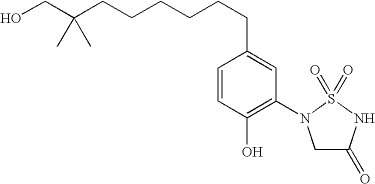

The title compound is prepared analogously to Example 42 starting with 6-bromohex-1-ene: MS (M−1)⁻=383.

### EXAMPLE 45

### 7-[4-Hydroxy-3-(1,1,4-trioxo-1,2,5-thiadiazolidin-2-yl)-phenyl]-2,2-dimethylheptanenitrile

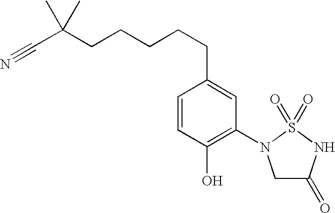

The title compound is prepared analogously to Example 35 from isobutyronitrile and 5-bromopent-1-ene: MS (M−1)⁻=364.

### EXAMPLE 46

### 5-[2-Hydroxy-5-(5-hydroxy-5-methylhex-1-ynyl)-phenyl]-1,1-dioxo-1,2,5-thiadiazolidin-3-one

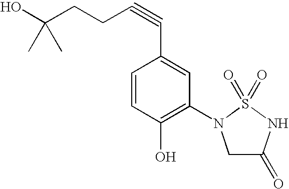

### A. Pent-4-ynoic acid methyl ester

To a solution of pent-4-ynoic acid (3 g, 30.61 mmol) in toluene (48 mL) and MeOH (12 mL) is added trimethylsilyldiazomethane (2 M in hexane, 16.07 mL, 32.14 mmol) dropwise and the mixture is stirred at ambient temperature for 3 h. 1N HCl is added dropwise and water is added. EtOAc is used to extract. The organic layer is washed with sat. NaHCO₃, brine and dried. Solvent is removed under reduced pressure and the residue is purified by column chromatography to give the title compound as a colorless oil.

### B. 2-Methylhex-5-yn-2-ol

To a solution of pent-4-ynoic acid methyl ester (440 mg, 3.9 mmol) in Et₂O (10 mL) is added methylmagnesium bromide (3 M in Et₂O, 5.2 mL, 15.6 mmol) dropwise and the mixture is stirred at ambient temperature for 3 h. The reaction mixture is then poured to a mixture of 1H HCl and Et₂O, and stirred vigorously. The ether layer is separated and washed with sat. NaHCO₃, brine and dried. The solvent is removed under reduced pressure to give the title compound as a pale yellow liquid.

### C. 5-[2-Hydroxy-5-(5-hydroxy-5-methylhex-1-ynyl)-phenyl]-1,1-dioxo-1,2,5-thiadiazolidin-3-one

The title compound is prepared analogously to Example 30, Step F, starting with 5-(5-bromo-2-hydroxyphenyl)-1,1-dioxo-1,2,5-thiadiazolidin-3-one and 2-methylhex-5-yn-2-ol, with the exception that Pd(dppf)Cl₂, CuCl and Et₃N is used in place of Pd(PPh₃)₄ and K₂CO₃: MS (M−1)⁻=364.

### EXAMPLE 47

### 5-[2-Hydroxy-5-(2-pyridin-3-yl-ethyl)-phenyl]-1,1-dioxo-1,2,5-thiadiazolidin-3-one

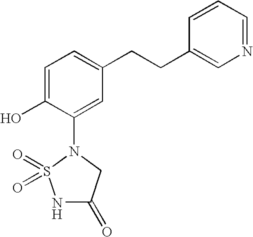

The title compound is prepared analogously to Example 35, Step B starting with 3-vinylpyridine, and Pd(OH)₂ is used in place of Pd/C for the debenzylation step: MS (M−1)⁻=332.

### EXAMPLE 48

### 5-(2-Hydroxy-4-methyl-5-pentylphenyl)-1,1-dioxo-1,2,5-thiadiazolidin-3-one

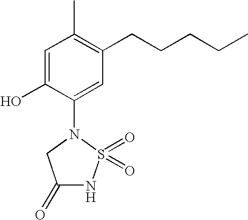

### A. 4-Benzyloxy-1-bromo-2-methylbenzene

The title compound is prepared analogously to Example 1, Step A from 4-bromo-3-methylphenol.

### B. 1-Benzyloxy-4-bromo-5-methyl-2-nitrobenzene

4-Benzyloxy-1-bromo-2-methylbenzene (13.4 g, 48.4 mmol) is dissolved in AcOH (100 mL) with heating and after it is cooled to RT, HNO₃ (65%, 4.4 mL, 96.8 mmol) is added dropwise. Concentrated H₂SO₄ (0.5 mL) is added and the mixture is heated to 70° C. More concentrated H₂SO₄ (0.5 mL) is added and the mixture is heated at 100° C. The mixture is then extracted with EtOAc and hexane and concentrated. The residue is purified by flash column chromatography (2% EtOAc/hexane) to give the title compound as a orange solid.

### C. 2-Benzyloxy-5-bromo-4-methylphenylamine

The title compound is prepared analogously to Example 1, Step B.

### D. 5-(2-Benzyloxy-5-bromo-4-methylphenyl)-1,1-dioxo-1,2,5-thiadiazolidin-3-one

The title compound is prepared analogously to Example 25, Steps D-G.

### E. 5-(2-Hydroxy-4-methyl-5-pentylphenyl)-1,1-dioxo-1,2,5-thiadiazolidin-3-one

The title compound is prepared analogously to Example 1, Steps F and G, using 1-pentenylboronic acid: Retention time=1.21 min (Method A) MS (M−1)⁻=311.

### EXAMPLE 49

The following compounds are prepared analogously to Example 48 using appropriate boronic acid.

| | | | Retention |
|---|---|---|---|
| | | | time |
| | | | (min) |
| Example | Chemical Name | MS (m/z) | Method |
| 49-1 | 5-(2-Hydroxy-4-methyl-5-propylphenyl)-1,1-dioxo- | (M − 1)⁻ = 283 | 1.01 |
| | 1,2,5-thiadiazolidin-3-one | | |
| 49-2 | 5-(5-Heptyl-2-hydroxy-4-methylphenyl)-1,1-dioxo- | (M − 1)⁻ = 339 | 1.39 |
| | 1,2,5-thiadiazolidin-3-one | | |
| 49-3 | 5-[5-(2-Cyclohexylethyl)-2-hydroxy-4-methylphenyl]- | (M − 1)⁻ = 351 | 1.49 |
| | 1,1-dioxo-1,2,5-thiadiazolidin-3-one | | |

### EXAMPLE 50

### Benzoic acid 4-(7-hydroxy-6,6-dimethylheptyl)-2-(1,1,4-trioxo-1,2,5-thiadiazolidin-2-yl)-phenyl ester

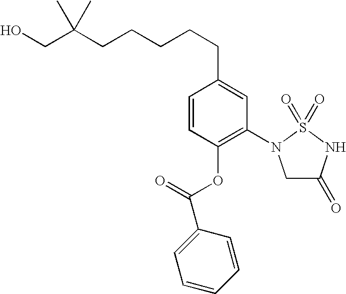

To the potassium salt of 5-[2-hydroxy-5-(7-hydroxy-6,6-dimethylheptyl)-phenyl]-1,1-dioxo-1,2,5-thiadiazolidin-3-one (Example 42) (320 mg, 0.78 mmol) in DMF (5 mL) at 0° C. is added KOtBu (1 M in THF, 0.78 mL, 0.78 mmol) dropwise. After it is stirred for 2 min., benzoyl chloride (0.090 mL, 0.78 mmol) is added dropwise. The mixture is stirred for 5 min. Water is added (5 drops) and the mixture is subjected to HPLC purification to isolate the title compound as a white solid: MS (M−1)⁻=473.

### EXAMPLE 51

### Benzoic acid 4-(6-cyano-6,6-dimethylhexyl)-2-(1,1,4-trioxo-1,2,5-thiadiazolidin-2-yl)-phenylester

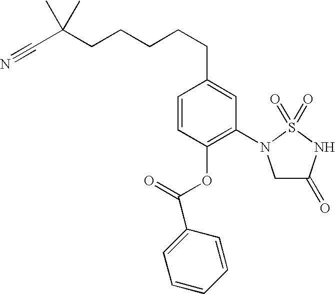

The title compound is prepared analogously to Example 50 starting from 8-[4-hydroxy-3-(1,1,4-trioxo-1,2,5-thiadiazolidin-2-yl)-phenyl]-2,2-dimethyloctanenitrile (Example 40): MS (M−1)⁻=378.

The table below shows the inhibitory activity (IC50 values) of representative compounds of the invention to human PTP-1B.

| Compound | IC50 (nM) |
|---|---|
| Example No. 2-5 | 80 nM |
| Example No. 13-7 | 86 nM |

1. A compound of the formula 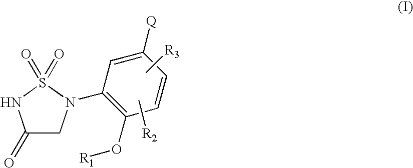
wherein
Q is alkoxy, alkylthio, alkylthiono, sulfonyl, cycloalkyl, aryl, aryloxy, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂ and R₃ are, independently from each other, hydrogen, halogen, (C₁₋₃)alkyl or (C₁₋₃)alkoxy;
or a pharmaceutically acceptable salt thereof.

2. The compound according to claim 1, wherein
Q is —Y—(CH₂)ₙ—CR₈R₉—(CH₂)ₘ—X in which
Y is oxygen or S(O)_{q} in which q is zero or an integer of 1 or 2; or
Y is C≡C; or
Y is absent;
n and m are, independently from each other, zero or an integer from 1 to 8;
R₈ and R₉ are, independently from each other, hydrogen or lower alkyl; or
R₈ and R₉ combined are alkylene which together with the carbon atom to which they are attached form a 3- to 7-membered ring;
X is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
or a pharmaceutically acceptable salt thereof.

3. The compound according to claim 2, wherein
R₂ and R₃ are hydrogen;
or a pharmaceutically acceptable salt thereof.

4. The compound according to claim 3, wherein
n is zero or an integer from 1 to 3;
m is zero or 1;
R₈ and R₉ are, independently from each other, hydrogen or lower alkyl;
X is hydroxy, carbamoyl, cyano, trifluoromethyl, free or esterified carboxy, heterocyclyl, monocyclic aryl or monocyclic aryloxy;
or a pharmaceutically acceptable salt thereof.

5. The compound according to claim 4, wherein
Y is C≡C; or
Y is absent;
or a pharmaceutically acceptable salt thereof.

6. The compound according to claim 5, wherein
Y is absent;
n is an integer of 5 or 6;
m is zero or 1;
R₈ and R₉ are lower alkyl;
X is hydroxy, cyano or free or esterified carboxy;
or a pharmaceutically acceptable salt thereof.

7. The compound according to claim 6, wherein
R₈ and R₉ are methyl;
or a pharmaceutically acceptable salt thereof.

8. The compound according to claim 7, wherein
R₁ is hydrogen or —C(O)R₄ in which R₄ is monocyclic aryl;
or a pharmaceutically acceptable salt thereof.

9. The compound according to claim 5, wherein
Y is absent;
n is an integer of 4 or 5;
m is zero;
R₈ and R₉ are hydrogen;
X is monocyclic aryloxy;
or a pharmaceutically acceptable salt thereof.

10. The compound according to claim 9, wherein
R₁ is hydrogen or —C(O)R₄ in which R₄ is monocyclic aryl;
or a pharmaceutically acceptable salt thereof.

11. The compound according to claim 5, wherein
Y is C≡C;
n is an integer of 2 or 3;
m is zero;
R₈ and R₉ are hydrogen;
X is hydroxy, cyano or free or esterified carboxy;
or a pharmaceutically acceptable salt thereof.

12. The compound according to claim 11, wherein
R₁ is hydrogen or —C(O)R₄ in which R₄ is monocyclic aryl;
or a pharmaceutically acceptable salt thereof.

13. The compound according to claim 1, wherein
Q is monocyclic aryl or 5- to 6-membered heterocyclic ring;
or a pharmaceutically acceptable salt thereof.

14. The compound according to claim 13, wherein
R₂ and R₃ are hydrogen;
or a pharmaceutically acceptable salt thereof.

15. The compound according to claim 14 of the formula 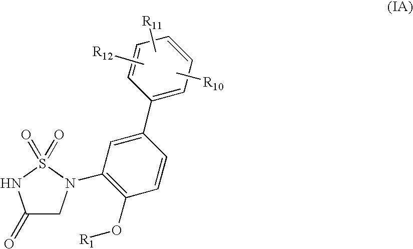
wherein
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₀, R₁₁ and R₁₂ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
C—R₁₀, C—R₁₁ and C—R₁₂ are, independently from each other, replaced by nitrogen;
or a pharmaceutically acceptable salt thereof.

16. The compound according to claim 15, wherein
R₁₀ and R₁₁ are hydrogen;
or a pharmaceutically acceptable salt thereof.

17. The compound according to claim 15, wherein
R₁ is hydrogen or —C(O)R₄ in which R₄ is monocyclic aryl;
or a pharmaceutically acceptable salt thereof.

18. The compound according to claim 14 of the formula 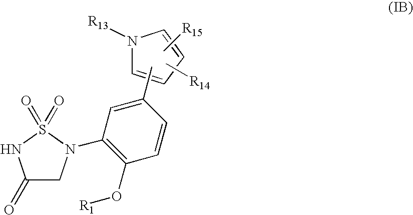
wherein
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁₄ and R₁₅ are, independently from each other, hydrogen or lower alkyl; or
C—R₁₄ and C—R₁₅ are, independently from each other, replaced by nitrogen;
or a pharmaceutically acceptable salt thereof.

19. The compound according to claim 18, wherein
C—R₁₄ is replaced by nitrogen;
R₁₅ is hydrogen;
or a pharmaceutically acceptable salt thereof.

20. The compound according to claim 19 of the formula 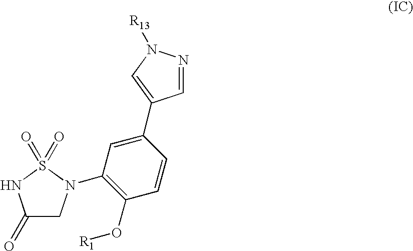
wherein
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
or a pharmaceutically acceptable salt thereof.

21. The compound according to claim 20, wherein
R₁₃ is —(CH₂)ₙ—CR₁₆R₁₇—(CH₂)ₘ-Z in which
n and m are, independently from each other, zero or an integer from 1 to 6;
R₁₆ and R₁₇ are, independently from each other, hydrogen or lower alkyl; or
R₁₆ and R₁₇ combined are alkylene which together with the carbon atom to which they are attached form a 3- to 7-membered ring;
Z is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
or a pharmaceutically acceptable salt thereof.

22. The compound according to claim 21, wherein
n is an integer from 1 to 3;
m is zero or 1;
R₁₆ and R₁₇ are, independently from each other, hydrogen or lower alkyl;
Z is hydroxy, carbamoyl, cyano, trifluoromethyl, free or esterified carboxy, heterocyclyl, monocyclic aryl or monocyclic aryloxy;
or a pharmaceutically acceptable salt thereof.

23. The compound according to claim 22, wherein
R₁₆ and R₁₇ are hydrogen;
Z is hydroxy, cyano or free or esterified carboxy;
or a pharmaceutically acceptable salt thereof.

24. The compound according to claim 23, wherein
R₁ is hydrogen or —C(O)R₄ in which R₄ is monocyclic aryl;
or a pharmaceutically acceptable salt thereof.

25. The compound according to claim 1 selected from the group consisting of:
5-[2-Hydroxy-5-(1H-pyrrol-2-yl)-phenyl]-1,1-dioxo-1,2,5-thiadiazolidin-3-one;
5-(4-Hydroxybiphenyl-3-yl)-1,1-dioxo-1,2,5-thiadiazolidin-3-one;
5-[2-Hydroxy-5-(2H-pyrazol-3-yl)-phenyl]-1,1-dioxo-1,2,5-thiadiazolidin-3-one;
5-[2-Hydroxy-5-(1-methyl-1H-pyrazol-4-yl)-phenyl]-1,1-dioxo-1,2,5-thiadiazolidin-3-one;
5-(5-Furan-3-yl-2-hydroxyphenyl)-1,1-dioxo-1,2,5-thiadiazolidin-3-one;
5-[2-Hydroxy-5-(1H-pyrazol-4-yl)-phenyl]-1,1-dioxo-1,2,5-thiadiazolidin-3-one;
5-(4′-Acetyl-4-hydroxybiphenyl-3-yl)-1,1-dioxo-1,2,5-thiadiazolidin-3-one;
5-(4′-Benzoyl-4-hydroxybiphenyl-3-yl)-1,1-dioxo-1,2,5-thiadiazolidin-3-one;
5-[2-Hydroxy-5-(1H-pyrrol-3-yl)-phenyl]-1,1-dioxo-1,2,5-thiadiazolidin-3-one;
Methanesulfonic acid 4′-hydroxy-3′-(1,1,4-trioxo-1,2,5-thiadiazolidin-2-yl)-biphenyl-3-yl ester;
5-(3′-Amino-4-hydroxybiphenyl-3-yl)-1,1-dioxo-1,2,5-thiadiazolidin-3-one;
5-(4-Hydroxy-2′-methylbiphenyl-3-yl)-1,1-dioxo-1,2,5-thiadiazolidin-3-one;
5-[2-Hydroxy-5-(1H-indol-2-yl)-phenyl]-1,1-dioxo-1,2,5-thiadiazolidin-3-one;
[4′-Hydroxy-3′-(1,1,4-trioxo-1,2,5-thiadiazolidin-2-yl)-biphenyl-3-yl]-acetonitrile;
4′-Hydroxy-3′-(1,1,4-trioxo-1,2,5-thiadiazolidin-2-yl)-biphenyl-3-carboxylic acid (2-cyanoethyl)-amide;
3-[4′-Hydroxy-3′-(1,1,4-trioxo-1,2,5-thiadiazolidin-2-yl)-biphenyl-3-yl]-propionic acid methyl ester;
4′-Hydroxy-3′-(1,1,4-trioxo-1,2,5-thiadiazolidin-2-yl)-biphenyl-3-carboxylic acid (2-carbamoylethyl)-amide;
5-[3′-(2-Aminoethyl)-4-hydroxybiphenyl-3-yl]-1,1-dioxo-1,2,5-thiadiazolidin-3-one;
5-(3′-Aminomethyl-4-hydroxybiphenyl-3-yl)-1,1-dioxo-1,2,5-thiadiazolidin-3-one;
5-(2-Hydroxy-5-pyridin-3-yl-phenyl)-1,1-dioxo-1,2,5-thiadiazolidin-3-one;
5-(4-Hydroxy-2′-methoxy-biphenyl-3-yl)-1,1-dioxo-1,2,5-thiadiazolidin-3-one;
5-(2-Hydroxy-5-pyridin-4-yl-phenyl)-1,1-dioxo-1,2,5-thiadiazolidin-3-one;
[4′-Hydroxy-3′-(1,1,4-trioxo-1,2,5-thiadiazolidin-2-yl)-biphenyl-4-yl]-acetic acid;
5-(4′-Chloro-4-hydroxybiphenyl-3-yl)-1,1-dioxo-1,2,5-thiadiazolidin-3-one;
5-(3′-Chloro-4-hydroxybiphenyl-3-yl)-1,1-dioxo-1,2,5-thiadiazolidin-3-one;
5-[2-Hydroxy-5-(6-methoxypyridin-3-yl)-phenyl]-1,1-dioxo-1,2,5-thiadiazolidin-3-one;
5-[5-(6-Fluoropyridin-3-yl)-2-hydroxyphenyl]-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionic acid ethyl ester;
5-(4-Hydroxy-3′-methylbiphenyl-3-yl)-1,1-dioxo-1,2,5-thiadiazolidin-3-one;
5-(3′-Fluoro-4-hydroxybiphenyl-3-yl)-1,1-dioxo-1,2,5-thiadiazolidin-3-one;
5-(4′-Fluoro-4-hydroxybiphenyl-3-yl-1,1-dioxo-1,2,5-thiadiazolidin-3-one;
5-(4-Hydroxy-4′-methylbiphenyl-3-yl)-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionitrile;
4′-Hydroxy-3′-(1,1,4-trioxo-1,2,5-thiadiazolidin-2-yl)-biphenyl-3-carbonitrile;
5-(4-Hydroxy-3′,5′-dimethylbiphenyl-3-yl)-1,1-dioxo-1,2,5-thiadiazolidin-3-one;
5-(4-Hydroxy-3′-methoxybiphenyl-3-yl)-1,1-dioxo-1,2,5-thiadiazolidin-3-one;
N-(2-Hydroxyethyl)-2-[4′-hydroxy-3′-(1,1,4-trioxo-1,2,5-thiadiazolidin-2-yl)-biphenyl-4-yl]-acetamide;
2,2,2-Trifluoro-N-[4′-hydroxy-3′-(1,1,4-trioxo-1,2,5-thiadiazolidin-2-yl)-biphenyl-3-yl]-acetamide;
1-Ethyl-3-[4′-hydroxy-3′-(1,1,4-trioxo-1,2,5-thiadiazolidin-2-yl)-biphenyl-3-yl]-urea;
1-Ethyl-3-[4′-hydroxy-3′-(1,1,4-trioxo-1,2,5-thiadiazolidin-2-yl)-biphenyl-3-ylmethyl]-urea;
[4′-Hydroxy-3′-(1,1,4-trioxo-1,2,5-thiadiazolidin-2-yl)-biphenyl-3-ylmethyl]-carbamic acid methyl ester;
N-[4′-Hydroxy-3′-(1,1,4-trioxo-1,2,5-thiadiazolidin-2-yl)-biphenyl-3-ylmethyl]-acetamide;
[4′-Hydroxy-3′-(1,1,4-trioxo-1,2,5-thiadiazolidin-2-yl)-biphenyl-3-ylmethyl]-carbamic acid benzyl ester;
1-Ethyl-3-[4′-hydroxy-3′-(1,1,4-trioxo-1,2,5-thiadiazolidin-2-yl)-biphenyl-4-yl]-urea;
3-[4′-Hydroxy-3′-(1,1,4-trioxo-1,2,5-thiadiazolidin-2-yl)-biphenyl-3-yl]-propionic acid;
5-{4-[4-Hydroxy-3-(1,1,4-trioxo-1,2,5-thiadiazolidin-2-yl)-phenyl]-pyrazol-1-yl}-pentanoic acid;
5-[2-Hydroxy-5-(1-propyl-1H-pyrazol-4-yl)-phenyl]-1,1-dioxo-1,2,5-thiadiazolidin-3-one;
5-[2-Hydroxy-5-(1-isobutyl-1H-pyrazol-4-yl)-phenyl]-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanoic acid ethyl ester;
5-{2-Hydroxy-5-[1-(4,4,4-trifluorobutyl)-1H-pyrazol-4-yl]-phenyl}-1,1-dioxo-1,2,5-thiadiazolidin-3-one;
5-{2-Hydroxy-5-[1-(3-methylbutyl)-1H-pyrazol-4-yl]-phenyl}-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanenitrile;
4-{4-[4-Hydroxy-3-(1,1,4-trioxo-1,2,5-thiadiazolidin-2-yl)-phenyl]-1H-pyrazol-1-yl}-butyronitrile;
5-(2-Hydroxy-5-phenoxyphenyl)-1,1-dioxo-1,2,5-thiadiazolidin-3-one;
5-(2-Hydroxy-5-methoxyphenyl)-1,1-dioxo-1,2,5-thiadiazolidin-3-one;
5-(5-Benzyl-2-hydroxyphenyl)-1,1-dioxo-1,2,5-thiadiazolidin-3-one;
5-(2-Hydroxy-5-methylphenyl)-1,1-dioxo-1,2,5-thiadiazolidin-3-one;
5-(5-Hexyl-2-hydroxyphenyl)-1,1-dioxo-1,2,5-thiadiazolidin-3-one;
5-(5-Butyl-2-hydroxyphenyl)-1,1-dioxo-1,2,5-thiadiazolidin-3-one;
5-[2-Hydroxy-5-(tetrahydrofuran-3-yl)-phenyl]-1,1-dioxo-1,2,5-thiadiazolidin-3-one;
5-[5-(4-Fluorophenylethynyl)-2-hydroxyphenyl]-1,1-dioxo-1,2,5-thiadiazolidin-3-one;
6-[4-Hydroxy-3-(1,1,4-trioxo-1,2,5-thiadiazolidin-2-yl)-phenyl]-hex-5-ynenitrile;
6-[4-Hydroxy-3-(1,1,4-trioxo-1,2,5-thiadiazolidin-2-yl)-phenyl]-hex-5-ynoic acid;
5-[5-(3,3-Dimethyl-but-1-ynyl)-2-hydroxyphenyl]-1,1-dioxo-1,2,5-thiadiazolidin-3-one;
5-[2-Hydroxy-5-(5-methylhexyl)-phenyl]-1,1-dioxo-1,2,5-thiadiazolidin-3-one;
6-[4-Hydroxy-3-(1,1,4-trioxo-1,2,5-thiadiazolidin-2-yl)-phenyl]-hexanoic acid;
5-[5-(Benzylaminomethyl)-2-hydroxyphenyl]-1,1-dioxo-1,2,5-thiadiazolidin-3-one;
5-(5-Butylaminomethyl-2-hydroxyphenyl)-1,1-dioxo-1,2,5-thiadiazolidin-3-one;
5-{2-Hydroxy-5-[(2-methoxybenzylamino)-methyl]-phenyl}-1,1-dioxo-1,2,5-thiadiazolidin-3-one;
5-{5-[(2-Ethoxybenzylamino)-methyl]-2-hydroxyphenyl}-1,1-dioxo-1,2,5-thiadiazolidin-3-one;
5-{2-Hydroxy-5-[(2-isopropoxybenzylamino)-methyl]-phenyl}-1,1-dioxo-1,2,5-thiadiazolidin-3-one;
5-(2-Hydroxy-5-{[2-(1-methyl-2-phenylethoxy)-benzylamino]-methyl}-phenyl)-1,1-dioxo-1,2,5-thiadiazolidin-3-one;
5-[2-Hydroxy-5-(3-methylbutoxy)-phenyl]-1,1-dioxo-1,2,5-thiadiazolidin-3-one;
5-[2-Hydroxy-5-(4-methylpentyloxy)-phenyl]-1,1-dioxo-1,2,5-thiadiazolidin-3-one;
5-(2-Hydroxy-5-propoxyphenyl)-1,1-dioxo-1,2,5-thiadiazolidin-3-one;
2-Hydroxy-6-{4-[4-hydroxy-3-(1,1,4-trioxo-1,2,5-thiadiazolidin-2-yl)-phenyl]-butoxy}-N,N-dimethylbenzamide;
2-Hydroxy-6-{5-[4-hydroxy-3-(1,1,4-trioxo-1,2,5-thiadiazolidin-2-yl)-phenyl]-pentyloxy}-N,N-dimethylbenzamide;
2-Hydroxy-6-{6-[4-hydroxy-3-(1,1,4-trioxo-1,2,5-thiadiazolidin-2-yl)-phenyl]-hexyloxy}-N,N-dimethylbenzamide;
2-Fluoro-6-{6-[4-hydroxy-3-(1,1,4-trioxo-1,2,5-thiadiazolidin-2-yl)-phenyl]-hexyloxy}-N,N-dimethylbenzamide;
2-Hydroxy-6-{7-[4-hydroxy-3-(1,1,4-trioxo-1,2,5-thiadiazolidin-2-yl)-phenyl]-heptyloxy}-N,N-dimethylbenzamide;
5-(4-Hydroxy-4′-hydroxymethylbiphenyl-3-yl)-1,1-dioxo-1,2,5-thiadiazolidin-3-one;
5-(2-Hydroxy-4,5-dimethylphenyl)-1,1-dioxo-1,2,5-thiadiazolidin-3-one;
5-[4-Hydroxy-3-(1,1,4-trioxo-1,2,5-thiadiazolidin-2-yl)-phenyl]-2,2-dimethylpentanoic acid;
8-[4-Hydroxy-3-(1,1,4-trioxo-1,2,5-thiadiazolidin-2-yl)-phenyl]-2,2-dimethyloctanoic acid ethyl ester;
8-[4-Hydroxy-3-(1,1,4-trioxo-1,2,5-thiadiazolidin-2-yl)-phenyl]-2,2-dimethyloctanoic acid;
7-[4-Hydroxy-3-(1,1,4-trioxo-1,2,5-thiadiazolidin-2-yl)-phenyl]-2,2-dimethylheptanoic acid;
6-[4-Hydroxy-3-(1,1,4-trioxo-1,2,5-thiadiazolidin-2-yl)-phenyl]-2,2-dimethylhexanoic acid;
7-[4-Hydroxy-3-(1,1,4-trioxo-1,2,5-thiadiazolidin-2-yl)-phenyl]-2,2-dimethylheptanoic acid ethyl ester;
8-[4-Hydroxy-3-(1,1,4-trioxo-1,2,5-thiadiazolidin-2-yl)-phenyl]-2,2-dimethyloctanenitrile;
5-[2-Hydroxy-5-(6-hydroxy-6-methylheptyl)-phenyl]-1,1-dioxo-1,2,5-thiadiazolidin-3-one;
5-[2-Hydroxy-5-(7-hydroxy-6,6-dimethylheptyl)-phenyl]-1,1-dioxo-1,2,5-thiadiazolidin-3-one;
5-[2-Hydroxy-5-(5-hydroxy-5-methylhexyl)-phenyl]-1,1-dioxo-1,2,5-thiadiazolidin-3-one;
5-[2-hydroxy-5-(8-hydroxy-7,7-dimethyloctyl)-phenyl]-1,1-dioxo-1,2,5-thiadiazolidin-3-one;
7-[4-Hydroxy-3-(1,1,4-trioxo-1,2,5-thiadiazolidin-2-yl)-phenyl]-2,2-dimethylheptanenitrile;
5-[2-Hydroxy-5-(5-hydroxy-5-methylhex-1-ynyl)-phenyl]-1,1-dioxo-1,2,5-thiadiazolidin-3-one;
5-[2-Hydroxy-5-(2-pyridin-3-yl-ethyl)-phenyl]-1,1-dioxo-1,2,5-thiadiazolidin-3-one;
5-(2-Hydroxy-4-methyl-5-pentylphenyl)-1,1-dioxo-1,2,5-thiadiazolidin-3-one;
5-(2-Hydroxy-4-methyl-5-propylphenyl)-1,1-dioxo-1,2,5-thiadiazolidin-3-one;
5-(5-Heptyl-2-hydroxy-4-methylphenyl)-1,1-dioxo-1,2,5-thiadiazolidin-3-one;
5-[5-(2-Cyclohexylethyl)-2-hydroxy-4-methylphenyl]-1,1-dioxo-1,2,5-thiadiazolidin-3-one;
Benzoic acid 4-(7-hydroxy-6,6-dimethylheptyl)-2-(1,1,4-trioxo-1,2,5-thiadiazolidin-2-yl)-phenyl ester; and
Benzoic acid 4-(6-cyano-6,6-dimethylhexyl)-2-(1,1,4-trioxo-1,2,5-thiadiazolidin-2-yl)-phenylester;
or a pharmaceutically acceptable salt thereof.

26. A method for the inhibition of PTPase activity, comprising:
administering to a mammal in need thereof a therapeutically effective amount of the compound of claim 1.

27-28. (canceled)

29. A method for the treatment of conditions mediated by PTP-1B activity in mammals, comprising:
administering to a mammal in need thereof a therapeutically effective amount of the compound of claim 1.

30. A method for modulating glucose levels in mammals, comprising:
administering to a mammal in need thereof a therapeutically effective amount of the compound of claim 1.

31. A method for the treatment of insulin resistance, glucose intolerance, type 2 diabetes, obesity, hypertension, ischemic diseases of the large and small blood vessels, dyslipidemia, atherosclerosis, vascular restenosis, irritable bowel syndrome, pancreatitis, cancer, osteoporosis, neurodegenerative diseases, infectious diseases, and diseases involving inflammation and the immune system, comprising:
administering to a mammal in need thereof a therapeutically effective amount of the compound of claim 1.

32. A pharmaceutical composition, comprising:
a therapeutically effective amount of the compound of claim 1 in combination with one or more pharmaceutically acceptable carriers.

33. (canceled)

34. A pharmaceutical composition, comprising:
a jointly therapeutically effective amount of the compound of claim 1 in combination with an anti-diabetic agents, a hypolipidemic agent, an anti-obesity agent or an anti-hypertensive agent.

35-40. (canceled)
